(19) United States
(12) Patent Application Publication
(10) Pub. No.: US 2015/0232837 A1
(43) Pub. Date: Aug. 20, 2015

(54) MIRNA MODULATORS OF CHRONIC VISCERAL INFLAMMATION

(71) Applicant: APTAMIR THERAPEUTICS, INC., Austin, TX (US)

(72) Inventor: Marc Thibonnier, Austin, TX (US)

(73) Assignee: APTAMIR THERAPEUTICS, INC., Austin, TX (US)

(21) Appl. No.: 14/424,512

(22) PCT Filed: Aug. 30, 2013
(86) PCT No.: PCT/US2013/057568

§ 371 Date: Feb. 27, 2015

Related U.S. Application Data
(60) Provisional application No. 61/695,471, filed on Aug. 31, 2012.

(51) Int. Cl. C12N 15/113; C12N 15/10; C12N 15/115

(52) U.S. Cl. CPC C12N 15/113; C12N 15/115; C12N 15/1048; C12N 2310/141; C12N 2310/16

(57) ABSTRACT

Provided are methods for the modulation of chronic visceral inflammation in a cell, tissue, organ and/or subject using miRNA agents. Also provided are miRNA agents (e.g., miRNA, agomirs, and antagomirs) that can modulate the level of chronic inflammation and methods of screening for such agents. Also provided are compositions (e.g., aptamirs or exomirs) that facilitate cell/tissue-specific delivery of the miRNA agents.

### CROSS REFERENCE TO RELATED APPLICATIONS

This application claims priority to U.S. Application Ser. No. 61/695,471, filed on Aug. 31, 2012, which is hereby incorporated by reference in its entirety.

### BACKGROUND OF THE INVENTION

A. Field of the Invention

The invention generally concerns compositions comprising microRNAs (miRNAs) and targeting agents, as well as methods for delivering a therapeutic composition comprising the same, and the use of these compositions to treat chronic visceral inflammation and related cardiometabolic and oncologic disorders.

B. Description of Related Art

Chronic low-grade inflammation and associated oxidative stress play a major role in the initiation, development and worsening of cardiovascular and metabolic disorders such atherosclerosis, dyslipidemia, visceral obesity, metabolic syndrome (characterized by a combination of blood pressure and blood glucose elevations, central obesity, elevated triglycerides and reduced HDL cholesterol), non-alcoholic fatty liver disease (NARD) (characterized by ectopic fat accumulation in the liver combined with a low-grade chronic inflammatory state resulting in abnormal glucose, fatty acid and lipoprotein metabolism, increased oxidative stress, deranged adipokine profiles. hypercoagulability, endothelial dysfunction, and accelerated atherosclerosis) and non-alcoholic steatohepatitis (NASH), a cause of liver fibrosis, cirrhosis, failure and cancer. Furthermore, chronic inflammation and associated oxidative stress play critical roles at different stages of tumor development, including initiation, promotion, malignant conversion, invasion, metastasis, immune surveillance and responses to therapy. Twenty percent of cancer burden is linked to obesity.

Until recently, adipose tissue was thought to be a passive repository of lipids, but it is now clear that visceral adipose tissue plays an active metabolic role and interacts with the immune system through inflammatory mediators and signaling molecules. Visceral adipose tissue (comprising pre-adipocytes, adipocytes, lymphocytes, macrophages. fibroblasts and vascular cells, see FIG. 1) not only stores and releases energy, but also is the source of many hormones, chemokines, cytokines and adipokines (known as the adipose inflammatory secretome), which trigger, amplify and sustain a chronic inflammatory state. In particular, the pro-inflammatory molecules secreted by adipose tissues recruit and activate macrophages and T cells, sustaining a cycle of worsening inflammation. The same adipokines and cytokines are also released into the systemic circulation, leading to insulin resistance in liver and skeletal muscle, endothelial damage, atherosclerotic lesions, and alteration in central nervous system hormonal and neuronal activity that affect energy expenditure and appetite.

There is currently no symptomatic or curative treatment targeting chronic visceral inflammation in human subjects. Accordingly, there is a need in the art for novel methods and compositions that modulate the inflammatory activity of visceral adipose tissue for use in the treatment or prevention of chronic visceral inflammation and related cardiometabolic and oncologic complications.

### SUMMARY OF THE INVENTION

In some aspects, disclosed herein are methods and compositions for the modulation of chronic visceral inflammation using microRNA (miRNA) agents and targeting agents. The methods of the invention generally involve the modulation of at least one inflammation regulator (e.g., a pro-inflammatory or anti-inflammatory molecule) in a cell, tissue, organ and/or subject using a miRNA agent. In some embodiments, modulation of inflammation is the reduction of inflammation. As used herein, the term “miRNA agent” refers to an oligonucleotide or oligonucleotide mimetic that directly or indirectly modulates inflammation. miRNA agents can act on a target gene or miRNA (e.g., by hybridizing with its target genes or miRNA sequences and consequently modulating their expression and activity). Conversely, an inflammatory target can act on a miRNA.

As used herein, the term “inflammation regulator” refers to a molecule (e.g., a polypeptide, protein, glycoprotein or the encoding nucleic acid) that regulates inflammation either directly or indirectly. The term encompasses pro- and anti-inflammatory molecules (e.g., chemokines, cytokines, hormones, and adipokines and regulators thereof). Exemplary inflammation regulators include, but are not limited to, AICDA, AIM2, AKT2, ANGPTL2 (angiopoietin-like 2), CASP1 (Caspase 1), CCL2 (Chemokine C-C motif ligand 2, MCP-1), CCL3 (MIP-1 alpha), CCL4 (MIP-1 beta), CCL5 (RANTES), CCL7 (MCP-3), CCL8 (MCP-2), CCL11 (Eotaxin), CCL15 (MIP-1 delta), CCL17 (TARC), CCL19 (MIP-3b), CCL20 (MIP-3-alpha), CCL22 (MDC), CCR2 (Chemokine C-C motif receptor 2, MCP-1-R), CCR7 (CD197), CD40LG (CD154), CD69, CEBPA, CEBPB, CFD (Adipsin), CHUK (IKKA), CXCR4 (Chemokine C-X-C motif receptor 4, CD184), MT-CO2 (Cytochrome c oxidase subunit II), CRP, CXCL1, CXCL2, CXCL3, CXCL5, CXCL9 (MIG), CXCL10 (IP-10), CXCL11 (ITAC), CXCL12 (SDF-1), CXCL13 (BCA1), CXCL16, FGA (Fibrinogen A), FGB (Fibrinogen B), FGG (Fibrinogen G), HIF1A, ICAM1 (CD54), IFNB1, IFNG, IKBKB (IKKB), IKBKE (IKKE), IL12A, IL12B, IL15, IL17A, IL18, IL1A, IL1B, IL2, IL23A, IL6, IL8 (CXCL8), INPP5D, IRAK1, IRAK2, IRF3 (Interferon-regulatory factor 3), IRF4 (Interferon-regulatory factor 4), IRF5 (Interferon-regulatory factor 5), LCN2 (Lipocalin 2), LEP (Leptin), MAP3K7, MMP2, MMP9, MYD88, NAMPT (Visfatin), NFKB1, NLRP3 (NLR family, pyrin domain containing 3), NOS2 (iNOS), SPP1 (Osteopontin), P2RX7 (Purinergic receptor P2X7), PDCD4, PKNOX1, PTX3 (Pentraxin 3), RBP4, RETN (Resistin), SAA1 (serum amyloid A1), SELE (E-selectin), SELP (P-selectin), SERPINE1 (plasminogen activator inhibitor type 1), SOCS1, SOCS3, SPI1, STAT1, TAB1, TAB2, TGFB1, TIRAP, TLR2 (CD282), TLR3 (CD283), TLR4 (CD284), TLR6 (CD286), TLR7, TLR8 (CD288), TNF (TNF alpha), TRAF6, TXNIP (Thioredoxin interacting protein), VCAM1, VEGFA, WNT5A, ADIPOQ (Adiponectin), AKT1, ALOX15, ARG1 (Arginase 1), ARG2 (Arginase 2), CCL16, CCL18, CCL24, CD36, CTBP1, CXCR1, CXCR2, FABP4, HDAC3, IL10, IL10RA (IL10R), IL13, IL1RN (IL1RA), IL3, IL33, IL4, IL4R, IL5, KDM6B (JMJD3), KLF2, KLF4, MAF (c-MAF), MRC1 (Macrophage mannose receptor 1), MT-COX3, MYC (c-Myc), NR1H3 (LXRA), PPARD, PPARG, PPARGC1A, PRDX3 (Peroxiredoxin), RETNLB (FIZZ1), SCARB1, SFRP5, SIRT1, SIRT2, SIRT3, SIRT4, SIRT5, SIRT6, SIRT7, SOCS2, STAT3, and STATE. As used herein, the term “cytokines” refers to small cell-signaling molecules (including proteins, peptides, and glycoproteins) that are secreted by cells, including immuno-modulating agents, such as interleukins and interferons.

In some aspects, provided are methods of modulating the inflammatory activity of a cell associated with chronic visceral inflammation, the method comprising contacting the cell with a miRNA agent that modulates the activity of at least one inflammation regulator in the cell. In some embodiments, the cell associated with chronic visceral inflammation is a pre-adipocyte, adipocyte, adipose tissue derived mesenchymal stem cell, stroma vascular cell, macrophage, lymphocyte, endothelial cell, vascular cell, fibroblast, hepatocyte, myocyte, or a precursor thereof. As used herein, the term “inflammatory activity” of a cell refers to the ability of the cell to promote or suppress inflammation in a tissue or subject, and the term “activity” of an inflammation regulator refers to any measurable biological activity including, without limitation, mRNA expression, protein expression, or inflammation modulating activity.

In some aspects, provided are methods of modulating chronic visceral inflammation in a tissue, the method comprising contacting the tissue with a miRNA agent that modulates activity of at least one inflammation regulator. In some embodiments, the tissue is brown fat, white fat, subcutaneous adipose tissue, visceral adipose tissue, liver or muscle.

In some aspects, provided are methods of treating chronic visceral inflammation in a human subject in need of treatment thereof, the method comprising administering to the human subject an effective amount of a miRNA agent that modulates activity of at least one inflammation regulator. Also disclosed are agomirs or antagomirs that modulate the activity of at least one inflammation regulator in a cell. As used herein, the term “agomir” refers to a synthetic oligonucleotide or oligonucleotide mimetic that functionally mimics a miRNA, and the term “antagomir” refers to a synthetic oligonucleotide or oligonucleotide mimetic having complementarity to a specific miRNA, and which inhibits the activity of that miRNA.

As used herein, the term “inflammation” refers to the biological response of cells, tissues to harmful stimuli, such as pathogens, damaged cells, toxic molecules or irritants. “Chronic inflammation” leads to a progressive shift in the type of cells present at the site of inflammation and is characterized by simultaneous destruction and healing of the tissue from the inflammatory process. “Chronic visceral inflammation” refers to the chronic inflammation associated with the visceral adipose tissue or fat that surrounds organs (e.g., stomach, large intestine, small intestine and other organs of the abdomen or gut).

The inflammation regulator may be any appropriate inflammation regulator or modulator. In some embodiments, the miRNA or miRNA agent directly binds to a target site of an inflammation regulator or modulator. In some embodiments, the miRNA agent directly binds to the mRNA or promoter region of at least one inflammation regulator. In some embodiments, the miRNA agent directly binds to the 5′UTR or coding sequence of the mRNA of at least one inflammation regulator. In some embodiments, the inflammation regulator or modulator is AICDA, AIM2, AKT2, ANGPTL2 (angiopoietin-like 2), CASP1 (Caspase 1), CCL2 (Chemokine C-C motif ligand 2, MCP-1), CCL3 (MIP-1 alpha), CCL4 (MIP-1 beta), CCL5 (RANTES), CCL7 (MCP-3), CCL8 (MCP-2), CCL11 (Eotaxin), CCL15 (MIP-1 delta), CCL17 (TARC), CCL19 (MIP-3b), CCL20 (MIP-3-alpha), CCL22 (MDC), CCR2 (Chemokine C-C motif receptor 2, MCP-1-R), CCR7 (CD197), CD40LG (CD154), CD69, CEBPA, CEBPB, CFD (Adipsin), CHUK (IKKA), CXCR4 (Chemokine C-X-C motif receptor 4, CD184), MT-CO2 (Cytochrome c oxidase subunit II), CRP, CXCL1, CXCL2, CXCL3, CXCL5, CXCL9 (MIG), CXCL10 (IP-10), CXCL11 (ITAC), CXCL12 (SDF-1), CXCL13 (BCA1), CXCL16, FGA (Fibrinogen A), FGB (Fibrinogen B), FGG (Fibrinogen G), HIF1A, ICAM1 (CD54), IFNB1, IFNG, IKBKB (IKKB), IKBKE (IKKE), IL12A, IL12B, IL15, IL17A, IL18, IL1A, IL1B, IL2, IL23A, IL6, IL8 (CXCL8), INPP5D, IRAK1, IRAK2, IRF3 (Interferon-regulatory factor 3), IRF4 (Interferon-regulatory factor 4), IRF5 (Interferon-regulatory factor 5), LCN2 (Lipocalin 2), LEP (Leptin), MAP3K7, MMP2, MMP9, MYD88, NAMPT (Visfatin), NFKB1, NLRP3 (NLR family, pyrin domain containing 3), NOS2 (iNOS), SPP1 (Osteopontin), P2RX7 (Purinergic receptor P2X7), PDCD4, PKNOX1, PTX3 (Pentraxin 3), RBP4, RETN (Resistin), SAA1 (serum amyloid A1), SELE (E-selectin), SELP (P-selectin), SERPINE1 (plasminogen activator inhibitor type 1), SOCS1, SOCS3, SPI1, STAT1, TAB1, TAB2, TGFB1, TIRAP, TLR2 (CD282), TLR3 (CD283), TLR4 (CD284), TLR6 (CD286), TLR7, TLR8 (CD288), TNF (TNF alpha), TRAF6, TXNIP (Thioredoxin interacting protein), VCAM1, VEGFA, WNT5A, ADIPOQ (Adiponectin), AKT1, ALOX15, ARG1 (Arginase 1), ARG2 (Arginase 2), CCL16, CCL18, CCL24, CD36, CTBP1, CXCR1, CXCR2, FABP4, HDAC3, IL10, IL10RA (IL10R), IL13, IL1RN (IL1RA), IL3, IL33, IL4, IL4R, IL5, KDM6B (JMJD3), KLF2, KLF4, MAF (c-MAF), MRC1 (Macrophage mannose receptor 1), MT-COX3, MYC (c-Myc), NR1H3 (LXRA), PPARD, PPARG, PPARGC1A, PRDX3 (Peroxiredoxin), RETNLB (FIZZ1), SCARB1, SFRP5, SIRT1, SIRT2, SIRT3, SIRT4, SIRT5, SIRT6, SIRT7, SOCS2, STAT3, and STATE.

In some embodiments, the inflammation regulator is a pro-inflammatory molecule and the miRNA agent downregulates the activity of the pro-inflammatory molecule. In some embodiments, the pro-inflammatory molecule is a pro-inflammatory cytokine As used herein, the terms “pro-inflammatory cytokine” refers to a cytokine, which promotes systemic inflammation. In some embodiments, the pro-inflammatory molecule is a positive regulator of a pro-inflammatory cytokine In some embodiments, the pro-inflammatory molecule is a negative regulator of anti-inflammatory cytokine

In some embodiments, the inflammation regulator is an anti-inflammatory molecule and the miRNA agent upregulates the activity of the anti-inflammatory molecule. In some embodiments, the anti-inflammatory molecule is an anti-inflammatory cytokine In some embodiments, the anti-inflammatory molecule is a positive regulator of an anti-inflammatory cytokine As used herein, the terms “anti-inflammatory cytokines” refers to those immuno-regulatory cytokines that counteracts at least one aspect of inflammation (e.g., cell activation or the production of pro-inflammatory cytokines) and thus contribute to the control of the magnitude of the inflammatory responses in vivo.

In some embodiments, the miRNA is a human miRNA and is selected from the group consisting of hsa-let-7a-5p, hsa-let-7b-5p, hsa-let-7c, hsa-let-7d-3p, hsa-let-7d-5p, hsa-let-7e-5p, hsa-let-7f-5p, hsa-let-7g-5p, hsa-let-7i-5p, hsa-miR-1, hsa-miR-10a-5p, hsa-miR-100-5p, hsa-miR-103a-2-5p, hsa-miR-103a-3p, hsa-miR-106a-5p, hsa-miR-106b-5p, hsa-miR-107, hsa-miR-122-5p, hsa-miR-124-3p, hsa-miR-1246, hsa-miR-1249, hsa-miR-125a-5p, hsa-miR-125b-5p, hsa-miR-126-3p, hsa-miR-1268a, hsa-miR-1275, hsa-miR-128-3p, hsa-miR-1281, hsa-miR-1307-3p, hsa-miR-130a-3p, hsa-miR-130b-3p, hsa-miR-132-3p, hsa-miR-132-5p, hsa-miR-133a-5p, hsa-miR-133b, hsa-miR-134-5p, hsa-miR-135a-5p, hsa-miR-138-5p, hsa-miR-139-3p, hsa-miR-140-3p, hsa-miR-143-3p, hsa-miR-145-5p, hsa-miR-1469, hsa-miR-146a-5p, hsa-miR-146b-5p, hsa-miR-147a, hsa-miR-148a-3p, hsa-miR-149-5p, hsa-miR-150-3p, hsa-miR-150-5p, hsa-miR-151a-3p, hsa-miR-151a-5p, hsa-miR-152-3p, hsa-miR-155-5p, hsa-miR-15a-5p, hsa-miR-15b-5p, hsa-miR-16-5p, hsa-miR-17-5p, hsa-miR-181a-3p, hsa-miR-181a-5p, hsa-miR-181b-3p, hsa-miR-181b-5p, hsa-miR-181c-5p, hsa-miR-182-5p, hsa-miR-183-5p, hsa-miR-185-5p, hsa-miR-186-5p, hsa-miR-188-5p, hsa-miR-190b, hsa-miR-191-5p, hsa-miR-1915-3p, hsa-miR-192-5p, hsa-miR-193a-5p, hsa-miR-193b-3p, hsa-miR-197-3p, hsa-miR-19a-3p, hsa-miR-19b-3p, hsa-miR-206, hsa-miR-20a-5p, hsa-miR-21-5p, hsa-miR-210-5p, hsa-miR-212-3p, hsa-miR-214-3p, hsa-miR-22-3p, hsa-miR-221-3p, hsa-miR-222-3p, hsa-miR-223-3p, hsa-miR-23a-3p, hsa-miR-23a-5p, hsa-miR-23b-3p, hsa-miR-24-2-5p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-296-5p, hsa-miR-299-5p, hsa-miR-29a-3p, hsa-miR-29b-3p, hsa-miR-29c-5p, hsa-miR-2c-3p, hsa-miR-30a-5p, hsa-miR-30b-5p, hsa-miR-30c-5p, hsa-miR-30d-5p, hsa-miR-31-5p, hsa-miR-320a, hsa-miR-320b, hsa-miR-320c, hsa-miR-320d, hsa-miR-323a-3p, hsa-miR-324-3p, hsa-miR-324-5p, hsa-miR-326, hsa-miR-328-3p, hsa-miR-330-3p, hsa-miR-331-3p, hsa-miR-337-3p, hsa-miR-337-5p, hsa-miR-339-3p, hsa-miR-339-5p, hsa-miR-33a-5p, hsa-miR-342-3p, hsa-miR-345-5p, hsa-miR-34a-5p, hsa-miR-361-5p, hsa-miR-362-5p, hsa-miR-369-3p, hsa-miR-370-3p, hsa-miR-371a-3p, hsa-miR-374a-5p, hsa-miR-378a-3p, hsa-miR-378a-5p, hsa-miR-421, hsa-miR-423-5p, hsa-miR-424-5p, hsa-miR-425-3p, hsa-miR-425-5p, hsa-miR-432-5p, hsa-miR-433-3p, hsa-miR-483-3p, hsa-miR-483-5p, hsa-miR-484, hsa-miR-485-3p, hsa-miR-487a-3p, hsa-miR-498, hsa-miR-500a-3p, hsa-miR-502-3p, hsa-miR-502-5p, hsa-miR-503-5p, hsa-miR-505-3p, hsa-miR-505-5p, hsa-miR-511-3p, hsa-miR-516a-3p, hsa-miR-517-5p, hsa-miR-517c-3p, hsa-miR-518e-3p, hsa-miR-518f-3p, hsa-miR-519b-3p, hsa-miR-519d-3p, hsa-miR-522-3p, hsa-miR-532-3p, hsa-miR-532-5p, hsa-miR-543, hsa-miR-550a-3p, hsa-miR-572, hsa-miR-573, hsa-miR-574-3p, hsa-miR-576-3p, hsa-miR-582-5p, hsa-miR-604, hsa-miR-610, hsa-miR-618, hsa-miR-622, hsa-miR-625-5p, hsa-miR-629-3p, hsa-miR-629-5p, hsa-miR-630, hsa-miR-638, hsa-miR-642a-5p, hsa-miR-643, hsa-miR-650, hsa-miR-652-3p, hsa-miR-656-3p, hsa-miR-663a, hsa-miR-671-3p, hsa-miR-671-5p, hsa-miR-7-5p, hsa-miR-744-5p, hsa-miR-766-3p, hsa-miR-875-5p, hsa-miR-877-5p, hsa-miR-885-5p, hsa-miR-9-5p, hsa-miR-92a-3p, hsa-miR-92b-3p, hsa-miR-93-5p, hsa-miR-940, hsa-miR-941, hsa-miR-942-5p, hsa-miR-96-5p, hsa-miR-99a-5p, and hsa-miR-99b-5p or an analog, agomir, or antagomir thereof. The sequences of these miRNAs are well known in the art and may be found, for example, on the world wide web at mirbase.org, and are incorporated herein by reference in their entirety.

In some embodiments, the miRNA is a human miRNA and is selected from the group consisting of hsa-let-7a-5p, hsa-let-7b-5p, hsa-let-7c, hsa-let-7d-3p, hsa-let-7d-5p, hsa-let-7e-5p, hsa-let-7f-5p, hsa-let-7g-5p, hsa-let-7i-5p, hsa-miR-1, hsa-miR-100-5p, hsa-miR-103a-2-5p, hsa-miR-103a-3p, hsa-miR-106a-5p, hsa-miR-106b-5p, hsa-miR-107, hsa-miR-10a-5p, hsa-miR-122-5p, hsa-miR-124-3p, hsa-miR-1246, hsa-miR-1249, hsa-miR-125a-5p, hsa-miR-125b-5p, hsa-miR-126-3p, hsa-miR-1268a, hsa-miR-1275, hsa-miR-130a-3p, hsa-miR-130b-3p, hsa-miR-132-3p, hsa-miR-132-5p, hsa-miR-133b, hsa-miR-135a-5p, hsa-miR-138-5p, hsa-miR-139-3p, hsa-miR-140-3p, hsa-miR-143-3p, hsa-miR-145-5p, hsa-miR-146a-5p, hsa-miR-146b-5p, hsa-miR-147a, hsa-miR-148a-3p, hsa-miR-149-5p, hsa-miR-150-3p, hsa-miR-150-5p, hsa-miR-151a-3p, hsa-miR-151a-5p, hsa-miR-155-5p, hsa-miR-15a-5p, hsa-miR-15b-5p, hsa-miR-16-5p, hsa-miR-17-5p, hsa-miR-181a-3p, hsa-miR-181a-5p, hsa-miR-181b-5p, hsa-miR-181c-5p, hsa-miR-182-5p, hsa-miR-183-5p, hsa-miR-185-5p, hsa-miR-186-5p, hsa-miR-188-5p, hsa-miR-190b, hsa-miR-191-5p, hsa-miR-1915-3p, hsa-miR-192-5p, hsa-miR-193a-5p, hsa-miR-193b-3p, hsa-miR-197-3p, hsa-miR-19a-3p, hsa-miR-19b-3p, hsa-miR-206, hsa-miR-20a-5p, hsa-miR-21-5p, hsa-miR-212-3p, hsa-miR-214-3p, hsa-miR-22-3p, hsa-miR-221-3p, hsa-miR-222-3p, hsa-miR-223-3p, hsa-miR-23a-3p, hsa-miR-23a-5p, hsa-miR-23b-3p, hsa-miR-24-2-5p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-296-5p, hsa-miR-299-5p, hsa-miR-29a-3p, hsa-miR-29b-3p, hsa-miR-29c-5p, hsa-miR-30a-5p, hsa-miR-30b-5p, hsa-miR-30c-5p, hsa-miR-30d-5p, hsa-miR-31-5p, hsa-miR-320a, hsa-miR-320b, hsa-miR-320c, hsa-miR-323a-3p, hsa-miR-324-3p, hsa-miR-324-5p, hsa-miR-326, hsa-miR-330-3p, hsa-miR-331-3p, hsa-miR-337-3p, hsa-miR-337-5p, hsa-miR-339-3p, hsa-miR-339-5p, hsa-miR-33a-5p, hsa-miR-342-3p, hsa-miR-345-5p, hsa-miR-34a-5p, hsa-miR-361-5p, hsa-miR-362-5p, hsa-miR-369-3p, hsa-miR-37 1a-3p, hsa-miR-374a-5p, hsa-miR-378a-3p, hsa-miR-378a-5p, hsa-miR-421, hsa-miR-423-5p, hsa-miR-424-5p, hsa-miR-425-3p, hsa-miR-425-5p, hsa-miR-432-5p, hsa-miR-483-3p, hsa-miR-483-5p, hsa-miR-484, hsa-miR-498, hsa-miR-500a-3p, hsa-miR-502-3p, hsa-miR-502-5p, hsa-miR-503-5p, hsa-miR-505-3p, hsa-miR-505-5p, hsa-miR-518e-3p, hsa-miR-518f-3p, hsa-miR-532-3p, hsa-miR-532-5p, hsa-miR-543, hsa-miR-550a-3p, hsa-miR-572, hsa-miR-574-3p, hsa-miR-576-3p, hsa-miR-582-5p, hsa-miR-618, hsa-miR-625-5p, hsa-miR-629-3p, hsa-miR-629-5p, hsa-miR-630, hsa-miR-638, hsa-miR-642a-5p, hsa-miR-643, hsa-miR-650, hsa-miR-652-3p, hsa-miR-663a, hsa-miR-671-5p, hsa-miR-7-5p, hsa-miR-744-5p, hsa-miR-877-5p, hsa-miR-9-5p, hsa-miR-92a-3p, hsa-miR-92b-3p, hsa-miR-93-5p, hsa-miR-940, hsa-miR-941, hsa-miR-96-5p, hsa-miR-99a-5p, and hsa-miR-99b-5p (which are expressed in both human adipocytes and macrophages) or an analog, agomir, or antagomir thereof. The sequences of these miRNAs are well known in the art and may be found, for example, on the world wide web at mirbase.org, and are incorporated herein by reference in their entirety.

In some embodiments, the miRNA is a human miRNA and is selected from the group consisting of hsa-let-7a-5p, hsa-let-7b-5p, hsa-let-7c, hsa-let-7e-5p, hsa-let-7f-5p, hsa-let-7g-5p, hsa-let-7i-5p, hsa-miR-106a-5p, hsa-miR-107, hsa-miR-125a-5p, hsa-miR-1275, hsa-miR-130a-3p, hsa-miR-130b-3p, hsa-miR-15a-5p, hsa-miR-15b-5p, hsa-miR-16-5p, hsa-miR-17-5p, hsa-miR-185-5p, hsa-miR-188-5p, hsa-miR-193a-5p, hsa-miR-206, hsa-miR-20a-5p, hsa-miR-296-5p, hsa-miR-30d-5p, hsa-miR-320a, hsa-miR-320b, hsa-miR-320c, hsa-miR-324-3p, hsa-miR-326, hsa-miR-330-3p, hsa-miR-331-3p, hsa-miR-339-5p, hsa-miR-369-3p, hsa-miR-423-5p, hsa-miR-424-5p, hsa-miR-484, hsa-miR-498, hsa-miR-532-3p, hsa-miR-532-5p, hsa-miR-543, hsa-miR-650, hsa-miR-671-5p, hsa-miR-93-5p, and hsa-miR-940 or an analog, agomir, or antagomir thereof. The sequences of these miRNAs are well known in the art and may be found, for example, on the world wide web at mirbase.org, and are incorporated herein by reference in their entirety.

In some embodiments, the miRNA is a human miRNA and is selected from the group consisting of hsa-let-7a-5p, hsa-let-7b-5p, hsa-let-7c, hsa-let-7e-5p, hsa-let-7f-5p, hsa-let-7i-5p, hsa-miR-1, hsa-miR-106a-5p, hsa-miR-107, hsa-miR-125a-5p, hsa-miR-1275, hsa-miR-130a-3p, hsa-miR-130b-3p, hsa-miR-140-3p, hsa-miR-146b-5p, hsa-miR-15a-5p, hsa-miR-15b-5p, hsa-miR-16-5p, hsa-miR-17-5p, hsa-miR-186-5p, hsa-miR-188-5p, hsa-miR-206, hsa-miR-20a-5p, hsa-miR-27a-3p, hsa-miR-27b-3p, hsa-miR-28-3p, hsa-miR-30b-5p, hsa-miR-30d-5p, hsa-miR-320a, hsa-miR-320b, hsa-miR-320c, hsa-miR-326, hsa-miR-330-3p, hsa-miR-342-3p, hsa-miR-361-5p, hsa-miR-362-5p, hsa-miR-423-5p, hsa-miR-424-5p, hsa-miR-498, hsa-miR-532-5p, hsa-miR-543, hsa-miR-618, hsa-miR-650, hsa-miR-671-5p, hsa-miR-93-5p, hsa-miR-940 or an analog, agomir, or antagomir thereof. The sequences of these miRNAs are well known in the art and may be found, for example, on the world wide web at mirbase.org, and are incorporated herein by reference in their entirety.

In some embodiments, the miRNA a human miRNA and is is selected from the group consisting of hsa-let-7a-5p, hsa-let-7b-5p, hsa-let-7c, hsa-let-7e-5p, hsa-let-7f-5p, hsa-let-7i-5p, hsa-miR-106a-5p, hsa-miR-107, hsa-miR-125a-5p, hsa-miR-1275, hsa-miR-130a-3p, hsa-miR-130b-3p, hsa-miR-15a-5p, hsa-miR-15b-5p, hsa-miR-16-5p, hsa-miR-17-5p, hsa-miR-188-5p, hsa-miR-206, hsa-miR-20a-5p, hsa-miR-30d-5p, hsa-miR-320a, hsa-miR-320b, hsa-miR-320c, hsa-miR-326, hsa-miR-330-3p, hsa-miR-423-5p, hsa-miR-424-5p, hsa-miR-498, hsa-miR-532-5p, hsa-miR-543, hsa-miR-650, hsa-miR-671-5p, hsa-miR-93-5p, and hsa-miR-940 or an analog, agomir, or antagomir thereof. The sequences of these miRNAs are well known in the art and may be found, for example, on the world wide web at mirbase.org, and are incorporated herein by reference in their entirety.

In some embodiments, the composition comprises 1, 2, 3, 4, 5, 6, 7, 8, 9, 10, or more distinct miRNA agents or any range derivable therein.

In some embodiments, the miRNA agent is linked to a targeting moiety or agent. In some embodiments, the targeting agent is an aptamer, an exosome, or a combination of an aptamer and an exosome.

Aptamers are usually single-stranded, short molecules of RNA, DNA or a nucleic acid analog, that may adopt three-dimensional conformations complementary to a wide variety of target molecules.

Exosomes are small membrane vesicles of endocytic origin that are secreted by many cell types. For example, exosomes may have a diameter of about 30 to about 100 nm. They may be formed by inward budding of the late endosome leading to the formation of vesicle-containing multivesicular bodies (MVB), which then fuse with the plasma membrane to release exosomes into the extracellular environment. Though their exact composition and content depends on cell type and disease state, exosomes all share certain characteristics. In some embodiments, the composition further comprises a nanoparticle, such as an exosome, wherein the nanoparticle has a diameter of no more than 100 nm. In some embodiments, the nanoparticle has a diameter of equal to or between about 30 nm and about 100 nm. In some embodiments, the miRNA agent(s) is(are) encapsulated by the nanoparticle. In some embodiments, the targeting agent is bound to the outside of the nanoparticle.

In some embodiments, the targeting moiety is an aptamer. In some embodiments, the miRNA-targeting moiety is an aptamir. As used herein, the term “aptamir” refers to the combination of an aptamer (oligonucleic acid or peptide molecule that bind to a specific target molecule) and an agomir or antagomir as defined above, which allows cell or tissue-specific delivery of the miRNA agents. In some embodiments, the miRNA-targeting moiety is an exomir. As used herein, the term “exomir” refers to an exosome-based aptamir where the targeting aptamers are anchored at the surface of exosomes containing a miRNA analog load, described as an “ExomiR”. Exemplary carriers include nanoparticles or exosomes.

In some embodiments, the miRNA agent is covalently coupled to the targeting agent. In some embodiments, the miRNA agent is non-covalently coupled to the targeting agent. In some embodiments, the miRNA agent is coupled to the targeting agent by a linker. In some embodiments, the linker is selected from the group consisting of: a polyalkylene glycol, polyethylene glycol, a dendrimer, a comb polymer, a biotinstreptavidin bridge, and a ribonucleic acid.

In some embodiments, the targeting moiety delivers the miRNA agent to a specific cell type or tissue. In some embodiments, the targeting moiety delivers the miRNA agent to a pre-adipocyte, adipocyte, adipose tissue derived mesenchymal stem cell, stroma vascular cell, macrophage, lymphocyte, endothelial cell, vascular cell, fibroblast, hepatocyte, myocyte, or a precursor thereof. In some embodiments, the targeting moiety binds to at least one cell surface molecule from the group including Alix (PDCD6IP), ANXA1 (Annexin 1), ANXA2 (Annexin 2), ANXA4 (Annexin 4), ANXA5 (Annexin 5), ANXA6 (Annexin 6), ANXA7 (Annexin 7), Caveolin 1 (CAV1), Caveolin 2 (CAV2), CD10 (MME), CD13 (ANPEP), CD146 (MCAM), CD151 (TSPAN24), CD166 (ALCAM), CD29 (ITGB1), CD36 (FAT), CD44 (Hyaluronate), CD49e (ITGA4), CD59, CD63, CD81 (TSPAN28), CD90 (THY1), CD91 (LRP1), DCN (Decorin), DPT (Dermatopontin), FABP4, GYPC (Glycophorin C), ITGA7 (integrin, alpha 7), Lamp1, Mfge8 (Lactadherin), NPR1, SLC27A3 (FATP3), Stomatin, SXT10 (Syntaxin 10), TSPAN3, TSPAN4, CCR5 (CD195), CCR7 (CD197), CD14, CD33, CD40 (TNFRSF5), CD68, CD80, CD86, CSF1R (CD115), EMR1, Eng (CD105, Endoglin), FCGR1A (CD64), FCGR2A (CD32), FCGR2B (CD32), FCGR3A (CD16a), FCGR3B (CD16b), FUT4 (CD15), IL15RA (CD215), IL1R1 (CD121a), ITGAL (CD11a, Integrin alpha L), ITGAM (CD11b, Integrin alpha M, Mac-1), ITGAX (CD11c, Integrin alpha X), Lamp2 (CD107b, Mac3), LGALS3 (MAC2), PTPRC (CD45), SLAMF7 (CD319), TLR2 (CD282), TLR4 (CD284), TNFRSF1B (CD120b), CD163, CD1A, CD1B, CD209, CD226, CD302 (DCL-1), CD68, CD93, CLEC4A (Dectin-1), FCER2 (CD23), IL1R2 (CD121b), IL27RA, and MRC1 (CD206).

“Treatment”, or “treating” as used herein, is defined as the application or administration of a therapeutic agent (e.g., a miRNA agent or vector or transgene encoding same) to a patient, or application or administration of a therapeutic agent to an isolated tissue or cell line from a patient, and includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Effective amount” or “therapeutically effective amount” or “pharmaceutically effective amount” means that amount which, when administered to a subject or patient for treating a disease, is sufficient to effect such treatment for the disease. In some embodiments, the subject is administered at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, or 100 mg/kg (or any range derivable therein).

The composition may be administered to (or taken by) the patient 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range derivable therein. It is specifically contemplated that the composition may be administered once daily, twice daily, three times daily, four times daily, five times daily, or six times daily (or any range derivable therein) and/or as needed to the patient. Alternatively, the composition may be administered every 2, 4, 6, 8, 12 or 24 hours (or any range derivable therein) to or by the patient.

In some embodiments, the compounds described herein are comprised in a pharmaceutical composition. In further embodiments, the compounds described herein and optional one or more additional active agents, can be optionally combined with one or more pharmaceutically acceptable excipients and formulated for administration via epidural, intraperitoneal, intramuscular, cutaneous, subcutaneous or intravenous injection. In some aspects, the compounds or the composition is administered by aerosol, infusion, or topical, nasal, oral, anal, ocular, or otic delivery. In further embodiments, the pharmaceutical composition is formulated for controlled release.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

Also disclosed are methods of screening for a miRNA agent that modulates inflammation, the method comprising a) providing an indicator cell comprising a human genome; b) contacting the indicator cell with a test miRNA agent; and c) determining the activity of at least one inflammation regulator in the indicator cell in the presence and absence of the miRNA agent, wherein a change in the activity of the inflammation regulator in the presence of the test miRNA agent identifies the test miRNA agent as a miRNA agent that modulates inflammation. In some embodiments, the cell is a pre-adipocyte, adipocyte, adipose tissue derived mesenchymal stem cell, stroma vascular cell, macrophage, lymphocyte, endothelial cell, vascular cell, fibroblast, hepatocyte, myocyte, or a precursor thereof. In some embodiments, the activity of the inflammation regulator determined in step (c) is the mRNA expression level protein expression level or inflammation modulating activity of the inflammation regulator.

A “subject” is a vertebrate, including any member of the class Mammalia, including humans, domestic and farm animals, and zoo, sports or pet animals, such as mouse, rabbit, pig, sheep, goat, cattle and higher primates.

The methods for producing the compounds described herein are not limited to the exemplary methods described herein. The compounds may be synthesized by any suitable method known in the art and it will be obvious to those skilled in the art that various adaptations, changes, modifications, substitutions, deletions or additions of procedures may be made without departing from the spirit and scope of the invention.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps, which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings illustrate by way of example and not limitation. For the sake of brevity and clarity, every feature of a given structure may not be labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 is a schematic representation of the different cellular and structural elements of adipose tissue.

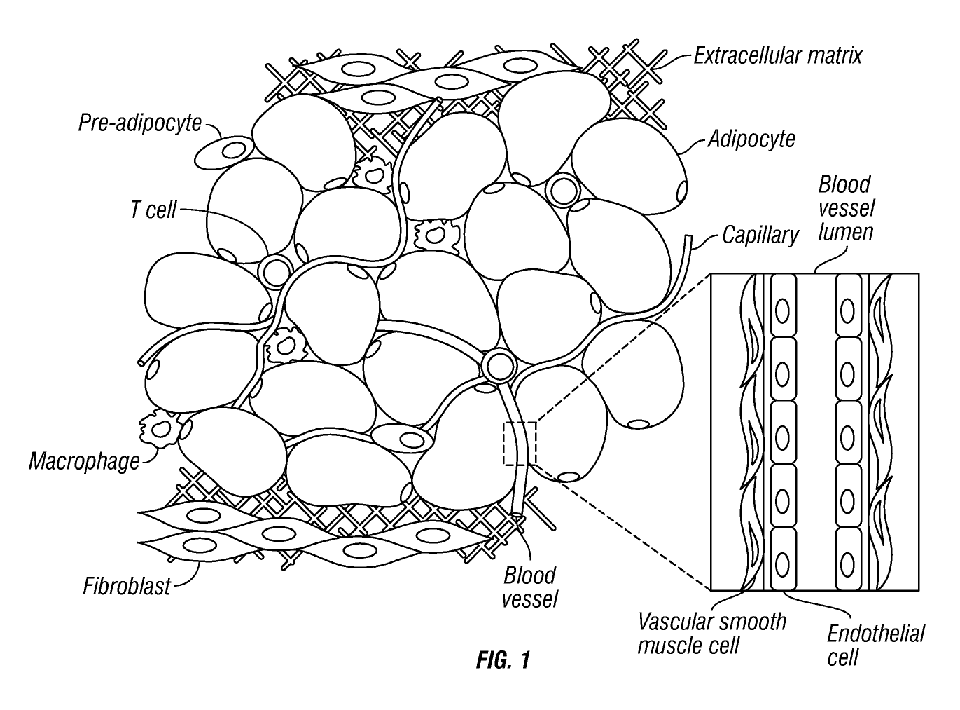

FIG. 2 is a schematic representation of the cellular alterations observed in adipose tissue during chronic inflammation.

FIG. 3 is a schematic representation of single-regulation, co-regulation, crosstalk, and independent gene regulatory networks (GRNs).

FIG. 4 is a schematic representation of the relationships between exemplary inflammation regulators.

FIG. 5 is a schematic representation of the functions of M1 and M2 macrophages and their distinct cytokine profile.

FIG. 6 is a schematic representation of the exemplary process for selecting cell specific aptamers.

### DETAILED DESCRIPTION OF THE INVENTION

In certain embodiments, the present invention provides methods for modulating chronic inflammation and its cardiovascular and metabolic complications. These methods generally involve contacting cells or tissue with a miRNA agent that modulates activity of at least one inflammation modulator. Such methods and compositions are particularly useful for treating inflammation (e.g., chronic visceral inflammation), atherosclerosis, dyslipidemias, visceral obesity, metabolic syndrome, non-alcoholic fatty liver disease (NAFLD), non-alcoholic steatohepatitis (NASH) and their related cancers.

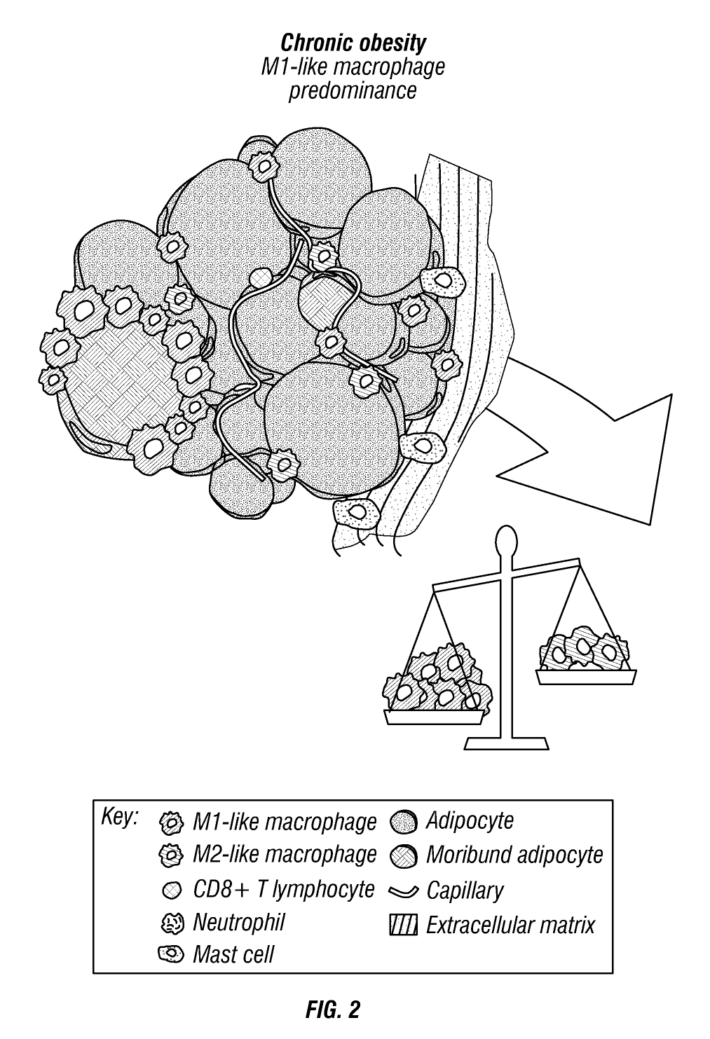

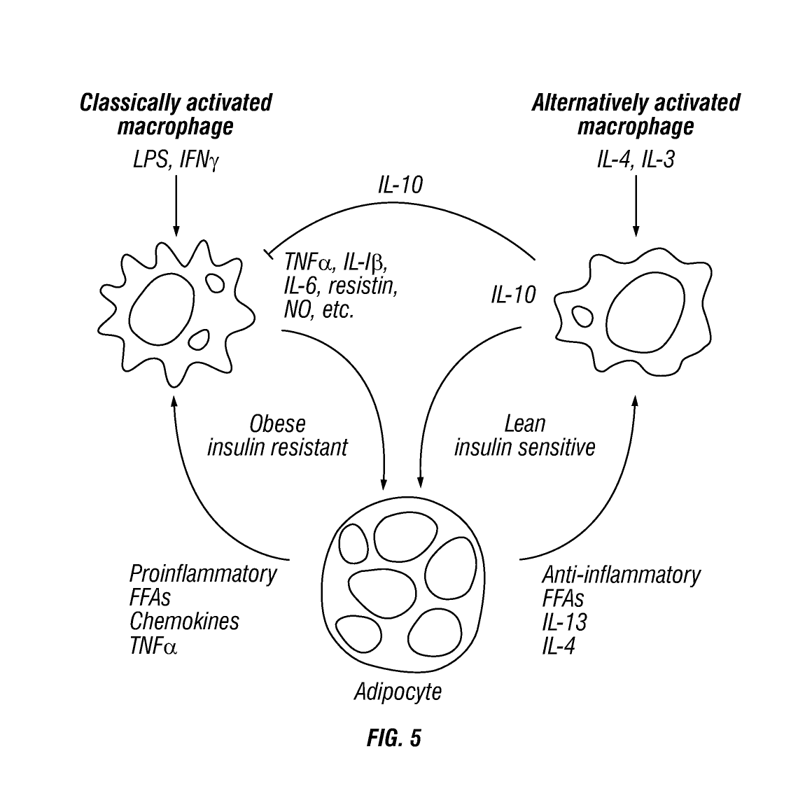

Inflammation is a component of multiple cardiovascular and metabolic risk factors and diseases. Pharmacological or genetic inhibition of pathways that underlie inflammatory responses has been found to protect experimental animals and human subjects from these risk factors and diseases. Macrophages are more abundant in adipose tissue of obese subjects than in adipose tissue of lean subjects (see FIG. 2) and appear to be a major source of inflammatory mediators released locally (paracrine effects) and systemically (endocrine effects). Under lean conditions, adipocytes secrete several factors that promote alternative activation of macrophages (see FIG. 5). Alternatively activated M2 macrophages secrete anti-inflammatory mediators, such as IL-10, and may secrete insulin-sensitizing factors. Obesity induces changes in adipocyte metabolism and gene expression, resulting in increased lipolysis and release of pro-inflammatory free fatty acids (FFAs) and factors that recruit and activate macrophages, such as monocyte chemotactic protein-1 (MCP-1 or CCL2) and tumor necrosis factor α (TNFα). Activated M1 macrophages produce large amounts of pro-inflammatory mediators, such as TNFα, IL-1β, IL-6 and resistin that act on adipocytes and other tissues/organs to induce an insulin-resistant state. Similarly, ectopic lipid accumulation in muscle, liver, and blood vessels activates tissue leukocytes, contributes to organ-specific disease, and exacerbates systemic insulin resistance.

Agents with anti-inflammatory actions can have beneficial effects by improving general health. However, most current anti-inflammatory therapeutic options have rather broad activities and alter innate immune functions. A more desirable approach would be to specifically inhibit the inflammatory components that have a more direct link to visceral inflammation. Accordingly, the invention provides novel methods and compositions for specifically modulating these inflammation regulators using miRNA agents.

In certain embodiments, miRNA agents are employed to downregulate the activity of a pro-inflammatory molecule (e.g., the mRNA expression level, protein expression level, or anti-inflammatory activity). As used herein, the terms “pro-inflammatory molecule” refers to a molecule (e.g., a polypeptide, protein, glycoprotein, or the encoding nucleic acid), which promotes systemic inflammation. Downregulation of a pro-inflammatory molecule can be achieved in several ways. In one embodiment, a miRNA agent directly inhibits the activity of a naturally occurring miRNA that is responsible for upregulation of the activity (e.g., the mRNA expression level, protein expression level) of the pro-inflammatory molecule. In another embodiment, the miRNA agent downregulates the activity (e.g., the mRNA expression level, protein expression level) of an activator of a pro-inflammatory molecule. This downregulation can be achieved, for example, by directly inhibiting the activity of a naturally occurring miRNA that is responsible for upregulation of the expression of the activator. In yet another embodiment, the miRNA agent upregulates the activity (e.g., the mRNA expression level, protein expression level) of a repressor of the pro-inflammatory molecule. This upregulation can be achieved, for example, by directly inhibiting the expression of a repressor of a pro-inflammatory molecule using a miRNA agent.

In certain embodiments, miRNA agents are employed to upregulate the activity of an anti-inflammatory molecule (e.g., the mRNA expression level, protein expression level, or anti-inflammatory activity). As used herein, the terms “anti-inflammatory molecule” refers to a molecule (e.g., a polypeptide, protein, glycoprotein, or the encoding nucleic acid) that counteracts at least one aspect of inflammation (e.g., cell activation or the production of pro-inflammatory cytokines) and thus contribute to the control of the magnitude of the inflammatory responses in vivo. Upregulation of an anti-inflammatory molecule can be achieved in several ways. In one embodiment, the miRNA agent directly inhibits the activity of a naturally occurring miRNA that is responsible for downregulation of the activity (e.g., the mRNA expression level, protein expression level) of the anti-inflammatory molecule. In another embodiment, the miRNA agent upregulates the activity (e.g., the mRNA expression level, protein expression level) of an activator of an anti-inflammatory molecule. This upregulation can be achieved, for example, by directly inhibiting the activity of a naturally occurring miRNA that is responsible for downregulation of the expression of the activator. In yet another embodiment, the miRNA agent downregulates the activity (e.g., the mRNA expression level or protein expression level) of a repressor of the anti-inflammatory molecule. This downregulation can be achieved, for example, by directly inhibiting the expression of a repressor of an anti-inflammatory molecule using a miRNA agent.

In certain embodiments, miRNA agents are employed that are capable of modulating the activity of multiple inflammation regulators simultaneously (pathway-specific miRNA agents as opposed to universal miRNA agents). For example, a single miRNA, agomir or antagomir that binds to multiple inflammation regulators can be used. This approach is particularly advantageous in that allows for the modulation of multiple members of an entire signaling pathway using a single miRNA agent.

In certain embodiments, multiple inhibitory miRNA agents (e.g., antagomirs or miR-masks) are employed. These inhibitory miRNA agents can have the same or different miRNA targets. As used herein, the term “miR-mask” refers to a single stranded antisense oligonucleotide that is complementary to a miRNA binding site in a target mRNA, and that serves to inhibit the binding of miRNA to the mRNA binding site. See, e.g., Xiao, et al., 2007, which is incorporated herein in its entirety.

### A. miRNA AGENTS

In certain embodiments, the invention employs miRNA agents for the modulation of inflammation regulators. miRNA agents suitable for use in the methods disclosed herein include, without limitation, miRNA, agomirs, antagomirs, miR-masks, miRNA-sponges, siRNA (single- or double-stranded), shRNA, antisense oligonucleotides, ribozymes, or other oligonucleotide mimetics which hybridize to at least a portion of a target nucleic acid and modulate its function.

In certain embodiments, the miRNA agents are miRNA molecules or synthetic derivatives thereof (e.g., agomirs). In one particular embodiment, the miRNA agent is a miRNA. miRNAs are a class of small (e.g., 18-24 nucleotides) non-coding RNAs that exist in a variety of organisms, including mammals, and are conserved in evolution. miRNAs are processed from hairpin precursors of about 70 nucleotides that are derived from primary transcripts through sequential cleavage by the RNAse III enzymes drosha and dicer. Many miRNAs can be encoded in intergenic regions, hosted within introns of pre-mRNAs or within ncRNA genes. Many miRNAs also tend to be clustered and transcribed as polycistrons and often have similar spatial temporal expression patterns. In general, miRNAs are post-transcriptional regulators that bind to complementary sequences on a target gene (mRNA or DNA), resulting in gene silencing by, e.g., translational repression or target degradation. One miRNA can target many different genes simultaneously. The interactions between miRNAs and their targets go beyond the original description of miRNAs as post-transcriptional regulators whose seed region of the driver strand (5′ bases 2-7) bind to complementary sequences in the 3′ UTR region of target mRNAs, usually resulting in translational repression or target degradation and gene silencing. The interactions can also involve various regions of the driver or passenger strands of the miRNAs as well as the 5′UTR, promoter, and coding regions of the mRNAs.

miRNAs are implicated in the inflammatory processes and oxidative stress that accompany heart failure, atherosclerosis, coronary artery disease, dyslipidemia, obesity, type 2 diabetes mellitus, cancer and the ageing process. Indeed, all ageing-associated diseases share the common denominator of chronic inflammation. miRNAs are involved in the regulation of adipocyte differentiation, oxidative stress, inflammation and angiogenesis in vascular and adipose tissues. Similarly, miRNAs regulate inflammation, oxidative stress, apoptosis, and angiogenesis in atherosclerotic plaques. miRNAs are potent regulator of inflammatory responses; for instance, miR-223 modulates macrophage polarization in a pattern that protects mice from diet-induced adipose tissue inflammation and systemic insulin resistance. miRNAs may contribute to communication between adipose and other tissues via circulating microvesicles and exosomes. As an example, miRNAs from visceral adipose tissue are differentially expressed in relation to the subtypes of NAFLD. These circulating miRNAs were found to be remarkably stable and to resist degradation from endogenous RNAse activity.

One miRNA can regulate hundreds of genes and one gene can be regulated by several miRNAs. miRNAs can potentially regulate the expression of many proteins. Many cytokines contain a miRNA target site in their 3′UTRs, with subsequent suppression of translation or degradation of their respective mRNA production. In addition, production of many cytokines can be indirectly controlled by miRNAs through interactions with adenine and uridine-rich elements-binding proteins (ARE-BPs), which positively or negatively regulate cytokine mRNA stability and/or translation. Finally, a number of cytokines regulate miRNA synthesis.

Accordingly, miRNAs are attractive drug candidates because the simultaneous modulation of many specific genes in targeted cells by a single miRNA can provide effective therapies for complex diseases like chronic low-grade inflammation, atherosclerosis, obesity, T2DM, metabolic syndrome, NAFLD, NASH and various cancers.

Exemplary miRNA molecules for use in the disclosed methods include those miRNA disclosed herein. In some embodiments, the miRNA molecules are one of 207 miRNAs involved in inflammation (the “InflaRNOme”). In particular, the InflaRNOme includes (miRBase Release 20 nomenclature) hsa-let-7a-5p, hsa-let-7b-5p, hsa-let-7c, hsa-let-7d-3p, hsa-let-7d-5p, hsa-let-7e-5p, hsa-let-7f-5p, hsa-let-7g-5p, hsa-let-7i-5p, hsa-miR-1, hsa-miR-10a-5p, hsa-miR-100-5p, hsa-miR-103a-2-5p, hsa-miR-103a-3p, hsa-miR-106a-5p, hsa-miR-106b-5p, hsa-miR-107, hsa-miR-122-5p, hsa-miR-124-3p, hsa-miR-1246, hsa-miR-1249, hsa-miR-125a-5p, hsa-miR-125b-5p, hsa-miR-126-3p, hsa-miR-1268a, hsa-miR-1275, hsa-miR-128-3p, hsa-miR-1281, hsa-miR-1307-3p, hsa-miR-130a-3p, hsa-miR-130b-3p, hsa-miR-132-3p, hsa-miR-132-5p, hsa-miR-133a-5p, hsa-miR-133b, hsa-miR-134-5p, hsa-miR-135a-5p, hsa-miR-138-5p, hsa-miR-139-3p, hsa-miR-140-3p, hsa-miR-143-3p, hsa-miR-145-5p, hsa-miR-1469, hsa-miR-146a-5p, hsa-miR-146b-5p, hsa-miR-147a, hsa-miR-148a-3p, hsa-miR-149-5p, hsa-miR-150-3p, hsa-miR-150-5p, hsa-miR-151a-3p, hsa-miR-151a-5p, hsa-miR-152-3p, hsa-miR-155-5p, hsa-miR-15a-5p, hsa-miR-15b-5p, hsa-miR-16-5p, hsa-miR-17-5p, hsa-miR-181a-3p, hsa-miR-181a-5p, hsa-miR-181b-3p, hsa-miR-181b-5p, hsa-miR-181c-5p, hsa-miR-182-5p, hsa-miR-183-5p, hsa-miR-185-5p, hsa-miR-186-5p, hsa-miR-188-5p, hsa-miR-190b, hsa-miR-191-5p, hsa-miR-1915-3p, hsa-miR-192-5p, hsa-miR-193a-5p, hsa-miR-193b-3p, hsa-miR-197-3p, hsa-miR-19a-3p, hsa-miR-19b-3p, hsa-miR-206, hsa-miR-20a-5p, hsa-miR-21-5p, hsa-miR-210-5p, hsa-miR-212-3p, hsa-miR-214-3p, hsa-miR-22-3p, hsa-miR-221-3p, hsa-miR-222-3p, hsa-miR-223-3p, hsa-miR-23a-3p, hsa-miR-23a-5p, hsa-miR-23b-3p, hsa-miR-24-2-5p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-296-5p, hsa-miR-299-5p, hsa-miR-29a-3p, hsa-miR-29b-3p, hsa-miR-29c-5p, hsa-miR-2c-3p, hsa-miR-30a-5p, hsa-miR-30b-5p, hsa-miR-30c-5p, hsa-miR-30d-5p, hsa-miR-31-5p, hsa-miR-320a, hsa-miR-320b, hsa-miR-320c, hsa-miR-320d, hsa-miR-323a-3p, hsa-miR-324-3p, hsa-miR-324-5p, hsa-miR-326, hsa-miR-328-3p, hsa-miR-330-3p, hsa-miR-331-3p, hsa-miR-337-3p, hsa-miR-337-5p, hsa-miR-339-3p, hsa-miR-339-5p, hsa-miR-33a-5p, hsa-miR-342-3p, hsa-miR-345-5p, hsa-miR-34a-5p, hsa-miR-361-5p, hsa-miR-362-5p, hsa-miR-369-3p, hsa-miR-370-3p, hsa-miR-371a-3p, hsa-miR-374a-5p, hsa-miR-378a-3p, hsa-miR-378a-5p, hsa-miR-421, hsa-miR-423-5p, hsa-miR-424-5p, hsa-miR-425-3p, hsa-miR-425-5p, hsa-miR-432-5p, hsa-miR-433-3p, hsa-miR-483-3p, hsa-miR-483-5p, hsa-miR-484, hsa-miR-485-3p, hsa-miR-487a-3p, hsa-miR-498, hsa-miR-500a-3p, hsa-miR-502-3p, hsa-miR-502-5p, hsa-miR-503-5p, hsa-miR-505-3p, hsa-miR-505-5p, hsa-miR-511-3p, hsa-miR-516a-3p, hsa-miR-517-5p, hsa-miR-517c-3p, hsa-miR-518e-3p, hsa-miR-518f-3p, hsa-miR-519b-3p, hsa-miR-519d-3p, hsa-miR-522-3p, hsa-miR-532-3p, hsa-miR-532-5p, hsa-miR-543, hsa-miR-550a-3p, hsa-miR-572, hsa-miR-573, hsa-miR-574-3p, hsa-miR-576-3p, hsa-miR-582-5p, hsa-miR-604, hsa-miR-610, hsa-miR-618, hsa-miR-622, hsa-miR-625-5p, hsa-miR-629-3p, hsa-miR-629-5p, hsa-miR-630, hsa-miR-638, hsa-miR-642a-5p, hsa-miR-643, hsa-miR-650, hsa-miR-652-3p, hsa-miR-656-3p, hsa-miR-663a, hsa-miR-671-3p, hsa-miR-671-5p, hsa-miR-7-5p, hsa-miR-744-5p, hsa-miR-766-3p, hsa-miR-875-5p, hsa-miR-877-5p, hsa-miR-885-5p, hsa-miR-9-5p, hsa-miR-92a-3p, hsa-miR-92b-3p, hsa-miR-93-5p, hsa-miR-940, hsa-miR-941, hsa-miR-942-5p, hsa-miR-96-5p, hsa-miR-99a-5p, and hsa-miR-99b-5p.

There are 673 miRNAs expressed in human adipocyte (“the AdipoRNOme” miRNAs). See International Application Serial No. PCT/US2013/037579 filed on Apr. 22, 2013, incorporated herein by reference in its entirety. The intersection between the InflaRNOme and the AdipoRNOme results in 174 miRNAs. In particular, the InflaRNOme includes (miRBase Release 20 nomenclature) includes hsa-let-7a-5p, hsa-let-7b-5p, hsa-let-7c, hsa-let-7d-3p, hsa-let-7d-5p, hsa-let-7e-5p, hsa-let-7f-5p, hsa-let-7g-5p, hsa-let-7i-5p, hsa-miR-1, hsa-miR-100-5p, hsa-miR-103a-2-5p, hsa-miR-103a-3p, hsa-miR-106a-5p, hsa-miR-106b-5p, hsa-miR-107, hsa-miR-10a-5p, hsa-miR-122-5p, hsa-miR-124-3p, hsa-miR-1246, hsa-miR-1249, hsa-miR-125a-5p, hsa-miR-125b-5p, hsa-miR-126-3p, hsa-miR-1268a, hsa-miR-1275, hsa-miR-130a-3p, hsa-miR-130b-3p, hsa-miR-132-3p, hsa-miR-132-5p, hsa-miR-133b, hsa-miR-135a-5p, hsa-miR-138-5p, hsa-miR-139-3p, hsa-miR-140-3p, hsa-miR-143-3p, hsa-miR-145-5p, hsa-miR-146a-5p, hsa-miR-146b-5p, hsa-miR-147a, hsa-miR-148a-3p, hsa-miR-149-5p, hsa-miR-150-3p, hsa-miR-150-5p, hsa-miR-151a-3p, hsa-miR-151a-5p, hsa-miR-155-5p, hsa-miR-15a-5p, hsa-miR-15b-5p, hsa-miR-16-5p, hsa-miR-17-5p, hsa-miR-181a-3p, hsa-miR-181a-5p, hsa-miR-181b-5p, hsa-miR-181c-5p, hsa-miR-182-5p, hsa-miR-183-5p, hsa-miR-185-5p, hsa-miR-186-5p, hsa-miR-188-5p, hsa-miR-190b, hsa-miR-191-5p, hsa-miR-1915-3p, hsa-miR-192-5p, hsa-miR-193a-5p, hsa-miR-193b-3p, hsa-miR-197-3p, hsa-miR-19a-3p, hsa-miR-19b-3p, hsa-miR-206, hsa-miR-20a-5p, hsa-miR-21-5p, hsa-miR-212-3p, hsa-miR-214-3p, hsa-miR-22-3p, hsa-miR-221-3p, hsa-miR-222-3p, hsa-miR-223-3p, hsa-miR-23a-3p, hsa-miR-23a-5p, hsa-miR-23b-3p, hsa-miR-24-2-5p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-296-5p, hsa-miR-299-5p, hsa-miR-29a-3p, hsa-miR-29b-3p, hsa-miR-29c-5p, hsa-miR-30a-5p, hsa-miR-30b-5p, hsa-miR-30c-5p, hsa-miR-30d-5p, hsa-miR-31-5p, hsa-miR-320a, hsa-miR-320b, hsa-miR-320c, hsa-miR-323a-3p, hsa-miR-324-3p, hsa-miR-324-5p, hsa-miR-326, hsa-miR-330-3p, hsa-miR-331-3p, hsa-miR-337-3p, hsa-miR-337-5p, hsa-miR-339-3p, hsa-miR-339-5p, hsa-miR-33a-5p, hsa-miR-342-3p, hsa-miR-345-5p, hsa-miR-34a-5p, hsa-miR-361-5p, hsa-miR-362-5p, hsa-miR-369-3p, hsa-miR-371a-3p, hsa-miR-374a-5p, hsa-miR-378a-3p, hsa-miR-378a-5p, hsa-miR-421, hsa-miR-423-5p, hsa-miR-424-5p, hsa-miR-425-3p, hsa-miR-425-5p, hsa-miR-432-5p, hsa-miR-483-3p, hsa-miR-483-5p, hsa-miR-484, hsa-miR-498, hsa-miR-500a-3p, hsa-miR-502-3p, hsa-miR-502-5p, hsa-miR-503-5p, hsa-miR-505-3p, hsa-miR-505-5p, hsa-miR-518e-3p, hsa-miR-518f-3p, hsa-miR-532-3p, hsa-miR-532-5p, hsa-miR-543, hsa-miR-550a-3p, hsa-miR-572, hsa-miR-574-3p, hsa-miR-576-3p, hsa-miR-582-5p, hsa-miR-618, hsa-miR-625-5p, hsa-miR-629-3p, hsa-miR-629-5p, hsa-miR-630, hsa-miR-638, hsa-miR-642a-5p, hsa-miR-643, hsa-miR-650, hsa-miR-652-3p, hsa-miR-663a, hsa-miR-671-5p, hsa-miR-7-5p, hsa-miR-744-5p, hsa-miR-877-5p, hsa-miR-9-5p, hsa-miR-92a-3p, hsa-miR-92b-3p, hsa-miR-93-5p, hsa-miR-940, hsa-miR-941, hsa-miR-96-5p, hsa-miR-99a-5p, and hsa-miR-99b-5p.

In certain embodiments, the invention employs miRNA analogs for the modulation of inflammation regulators. miRNA analogs, suitable for use in the methods disclosed herein, included, without limitation, miRNA, agomirs, antagomirs, miR-masks, miRNA-sponges, siRNA (single- or double-stranded), shRNA, antisense oligonucleotides, ribozymes, or other oligonucleotide mimetics which hybridize to at least a portion of a target nucleic acid and modulate its function. As used herein, the term “inflammation regulator” refers to a protein (or the encoding nucleic acid) that regulates inflammation either directly or indirectly. miRNA analogs such as agomirs are readily identifiable by a person skill in the art using the screening methods disclosed herein.

The term “agomir” refers to a synthetic oligonucleotide or oligonucleotide mimetic that functionally mimics a miRNA. An agomir can be an oligonucleotide with the same or similar nucleic acid sequence to a miRNA or a portion of a miRNA. In certain embodiments, the agomir has 1, 2, 3, 4, 5, 6, 7, 8, 9 or 10 nucleotide differences from the miRNA that it mimics. Further, agomirs can have the same length, a longer length or a shorter length than the miRNA that it mimics. In certain embodiments, the agomir has the same sequence as 6-8 nucleotides at the 5′ end of the miRNA it mimics. In other embodiments, an agomir can be 5, 6, 7, 8, 9, 10, 11, 12, 13, 14, 15, 16, 17, 18, 19, 20, 21, 22, 23, 24, 25, 27, 28, 29, 30, 31, 32, 33, 34, 35, 36, 37, 38, 39, 40, 41, 42, 43, 44, 45, 46, 47, 48, 49 or 50 nucleotides in length or any range derivable therein. In other embodiments, an agomir can be 5-10, 6-8, 10-20, 10-15 or 5-500 nucleotides in length or any range derivable therein. In certain embodiments, agomirs include any of the sequences of miRNAs disclosed herein. These chemically modified synthetic RNA duplexes include a guide strand that is identical or substantially identical to the miRNA of interest to allow efficient loading into the miRISC complex, whereas the passenger strand is chemically modified to prevent its loading to the Argonaute protein in the miRISC complex (Thorsen, et al., 2012; Broderick, et al., 2011).

The term “antagomir” refers to a synthetic oligonucleotide or oligonucleotide mimetic having complementarity to a specific microRNA, and which inhibits the activity of that miRNA. The term “antimir” is synonymous with the term “antagomir”. In certain embodiments, the antagomir has 1, 2, 3, 4, 5, 6, 7, 8, 9 or 10 nucleotide differences from the miRNA that it inhibits. Further, antagomirs can have the same length, a longer length or a shorter length than the miRNA that it inhibits. In certain embodiments, the antagomir hybridizes to 6-8 nucleotides at the 5′ end of the miRNA it inhibits. In other embodiments, an antagomir can be 5, 6, 7, 8, 9, 10, 11, 12, 13, 14, 15, 16, 17, 18, 19, 20, 21, 22, 23, 24, 25, 27, 28, 29, 30, 31, 32, 33, 34, 35, 36, 37, 38, 39, 40, 41, 42, 43, 44, 45, 46, 47, 48, 49 or 50 nucleotides in length or any range derivable therein. In other embodiments, an antagomir can be 5-10, 6-8, 10-20, 10-15 or 5-500 nucleotides in length or any range derivable therein. In certain embodiments, antagomirs include nucleotides that are complementary to any of the sequences of miRNAs disclosed herein. The antagomirs are synthetic reverse complements that tightly bind to and inactivate a specific miRNA. Various chemical modifications are used to improve nuclease resistance and binding affinity. The most commonly used modifications to increase potency include various 2′sugar modifications, such as 2′-O-Me, 2′-O-methoxyethyl (2′-MOE), or 2′-fluoro (2′-F). The nucleic acid structure of the miRNA can also be modified into a locked nucleic acid (LNA) with a methylene bridge between the 2′oxygen and the 4′ carbon to lock the ribose in the 3′-endo (North) conformation in the A-type conformation of nucleic acids (Lennox, et al., 2011; Bader, et al. 2011). This modification significantly increases both target specificity and hybridization properties of the molecules.

In certain embodiments, the miRNA analogs are oligonucleotide or oligonucleotide mimetics that inhibit the activity of one or more miRNA. Examples of such molecules include, without limitation, antagomirs, interfering RNA, antisense oligonucleotides, ribozymes, miRNA sponges and miR-masks. As used herein, the term “miRNA sponge” refers to a synthetic nucleic acid (e.g. a mRNA transcript) that contains multiple tandem-binding sites for a miRNA of interest, and that serves to titrate out the endogenous miRNA of interest, thus inhibiting the binding of the miRNA of interest to its endogenous targets. See, e.g., Ebert, et al., 2007, which is incorporated herein in its entirety. As used herein, the term “antisense oligonucleotide” refers to a synthetic oligonucleotide or oligonucleotide mimetic that is complementary to a DNA or mRNA sequence (e.g., an miRNA). In one particular embodiment, the miRNA analog is an antagomir. In general, antagomirs are chemically modified antisense oligonucleotides that bind to a target miRNA and inhibit miRNA function by prevent binding of the miRNA to its cognate gene target. Antagomirs can include any base modification known in the art. In one particular embodiment, the antagomir inhibits the activity of human miR-22 (van Rooij, et al., 2012; Snead, et al., 2012; Czech, et al., 2011).

In certain embodiments, the miRNA analogs are 10 to 50 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range derivable there within.

In certain embodiments, the miRNA agents are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference in its entirety.

In certain embodiments, the miRNA agents comprise at least one nucleotide modified at the 2′ position of the sugar, most preferably a 2′-0-alkyl, 2′-0-alkyl-0-alkyl or 2′-fluoro-modified nucleotide. In other preferred embodiments, RNA modifications include 2′-fluoro, 2′-amino and 2′ O-methyl modifications on the ribose of pyrimidines, a basic residue or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make an oligonucleotide more resistant to nuclease digestion, thereby prolonging in vivo half-life. Specific examples of modified oligonucleotides include those comprising backbones comprising, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)˜0˜CH2 (known as a methylene(methylimino) or MMI backbone], CH2-O—N(CH3)-CH2, CH2-N (CH3)-N(CH3)-CH2 and O—N (CH3)-CH2-CH2 backbones, wherein the native phosphodiester backbone is represented as O—P—O—CH,); amide backbones (see De Mesmaeker, et al., 1995); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen, et al, 1991), each of which is herein incorporated by reference in its entirety. Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2; see U.S. Pat. Nos. 3,687,808; 4,469,863; 4,476,301; 5,023,243; 5, 177,196; 5,188,897; 5,264,423; 5,276,019; 5,278,302; 5,286,717; 5,321, 131; 5,399,676; 5,405,939; 5,453,496; 5,455, 233; 5,466,677; 5,476,925; 5,519,126; 5,536,821; 5,541,306; 5,550,111; 5,563,253; 5,571,799; 5,587,361; and 5,625,050, each of which is herein incorporated by reference in its entirety. Morpholino-based oligomeric compounds are described in Braasch & Corey, 2002; Genesis 30:3, 2001; Heasman, 2002; Nasevicius et al, 2000; Lacerra et al, 2000; and U.S. Pat. No. 5,034,506, issued Jul. 23, 1991, each of which is herein incorporated by reference in its entirety. Cyclohexenyl nucleic acid oligonucleotide mimetics are described in Wang et al, 2000, the contents of which is incorporated herein in its entirety.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5, 185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference in its entirety.

In certain embodiments, miRNA agents comprise one or more substituted sugar moieties, e.g., one of the following at the 2′ position: OH, SH, SCH₃, F, OCN, OCH₃OCH₃, OCH₃O(CH₂)nCH₃, O(CH₂)nNH₂ or O(CH₂)nCH₃ where n is from 1 to about 10; Ci to CIO lower alkyl, alkoxyalkoxy, substituted lower alkyl, alkaryl or aralkyl; CI; Br; CN; CF3; OCF3; O—, S—, or N-alkyl; O—, S—, or N-alkenyl; SOCH3; SO2CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacokinetic/pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, 1995). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

In certain embodiments, miRNA agents comprise one or more base modifications and/or substitutions. As used herein, “unmodified” or “natural” bases include adenine (A), guanine (G), thymine (T), cytosine (C) and uracil (U). Modified bases include, without limitation, bases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic bases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, 1980; Gebeyehu, et al., 1987). A “universal” base known in the art, e.g., inosine, can also be included. 5-Me-C substitutions can also be included. These have been shown to increase nucleic acid duplex stability by 0.6-1.2° C. (Sanghvi, 1993). Further suitable modified bases are described in U.S. Pat. Nos. 3,687,808, as well as 4,845,205; 5,130,302; 5,134,066; 5,175, 273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

It is not necessary for all positions in a given oligonucleotide to be uniformly modified, and in fact more than one of the aforementioned modifications may be incorporated in a single oligonucleotide or even at within a single nucleoside within an oligonucleotide.

In certain embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, 1991.

In certain embodiments, the miRNA agent is linked (covalently or non-covalently) to one or more moieties or conjugates that enhance the activity, cellular distribution, or cellular uptake of the oligonucleotide. Such moieties include, without limitation, lipid moieties such as a cholesterol moiety (Letsinger et al, 1989), cholic acid (Manoharan et al, 1994), a thioether, e.g., hexyl-S-tritylthiol (Manoharan et al., 1992; Manoharan et al., 1993), a thiocholesterol (Oberhauser et al., 1992), an aliphatic chain, e.g., dodecandiol or undecyl residues (Kabanov et al, 1990; Svinarchuk et al., 1993), a phospholipid, e.g., di-hexadecyl-rac-glycerol or triethylammonium 1,2-di-O-hexadecyl-rac-glycero-3-H-phosphonate (Manoharan et al., 1995; Shea et al., 1990), a polyamine or a polyethylene glycol chain (Mancharan et al., 1995), or adamantane acetic acid (Manoharan et al., 1995), a palmityl moiety (Mishra et al., 1995), or an octadecylamine or hexylamino-carbonyl-toxycholesterol moiety (Crooke et al., 1996), each of which is herein incorporated by reference in its entirety. See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599,928 and 5,688,941, each of which is herein incorporated by reference in its entirety.

In some embodiments, the miRNA agent is linked to a targeting moiety or agent. In some embodiments, the targeting agent is an aptamer, an exosome, or a combination of an aptamer and an exosome. In one particular embodiment, the miRNA agent is linked to (covalently or non-covalently) a nucleic acid aptamer. Aptamers are synthetic oligonucleotides or peptide molecules that bind to a specific target molecule. In some embodiments, the miRNA-targeting moiety is an aptamir. As used herein, the term “aptamir” refers to the combination of an aptamer (oligonucleic acid or peptide molecule that bind to a specific target molecule) and an agomir or antagomir as defined above, which allows cell or tissue-specific delivery of the miRNA agents. In some embodiments, the miRNA-targeting moiety is an exomir. As used herein, the term “exomir” refers to an aptamer that is combined with a miRNA analog in the form of a carrier-based aptamiR, described as an “ExomiR”. Exemplary carriers include nanoparticles or exosomes. For a full discussion of aptamir and exomir compositions, see International Application Serial No. PCT/US2013/053613 filed on August 5, 2013, incorporated herein by reference in its entirety.

The miRNA agents must be sufficiently complementary to the target mRNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of a miRNA agent is capable of hydrogen bonding with a base at the corresponding position of a target nucleic acid sequence, then the bases are considered to be complementary to each other at that position. There may be 50, 55, 60, 65, 70, 75, 80, 85, 90, 95, or 100% complementarity. In certain embodiments, 100% complementarity is not required. In other embodiments, 100% complementarity is required.

miRNA agents for use in the methods disclosed herein can be designed using routine methods. While the specific sequences of certain exemplary target nucleic acid sequences and miRNA agents are set forth herein, one of skill in the art will recognize that these serve to illustrate and describe particular embodiments within the scope of the present invention. Additional target segments are readily identifiable by one having ordinary skill in the art in view of this disclosure. Target segments of 5, 6, 7, 8, 9, 10 or more nucleotides in length comprising a stretch of at least five (5) consecutive nucleotides within the seed sequence, or immediately adjacent thereto, are considered to be suitable for targeting a gene. In some embodiments, target segments can include sequences that comprise at least the 5 consecutive nucleotides from the 5′-terminus of one of the seed sequence (the remaining nucleotides being a consecutive stretch of the same RNA beginning immediately upstream of the 5’-terminus of the seed sequence and continuing until the miRNA agent contains about 5 to about 30 nucleotides). In some embodiments, target segments are represented by RNA sequences that comprise at least the 5 consecutive nucleotides from the 3′-terminus of one of the seed sequence (the remaining nucleotides being a consecutive stretch of the same miRNA beginning immediately downstream of the 3′-terminus of the target segment and continuing until the miRNA agent contains about 5 to about 30 nucleotides). One having skill in the art armed with the sequences provided herein will be able, without undue experimentation, to identify further preferred regions to target using miRNA agents. Once one or more target regions, segments or sites have been identified, inhibitory nucleic acid compounds are chosen that are sufficiently complementary to the target, i.e., that hybridize sufficiently well and with sufficient specificity (i.e., do not substantially bind to other non-target nucleic acid sequences), to give the desired effect.

In certain embodiments, miRNA agents used to practice this invention are expressed from a recombinant vector. Suitable recombinant vectors include, without limitation, DNA plasmids, viral vectors or DNA minicircles. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al., 1989; Coffin et al., 1997; Cann, 2000. As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors can be delivered as described herein, and persist in target cells (e.g., stable transformants).

In certain embodiments, miRNA agents used to practice this invention are synthesized in vitro using chemical synthesis techniques, as described in, e.g., Adams, 1983; Belousov, 1997; Frenkel, 1995; Blommers, 1994; Narang, 1979; Brown, 1979; Beaucage, 1981; U.S. Pat. No. 4,458,066, each of which is herein incorporated by reference in its entirety.

### B. TARGETED DELIVERY OF miRNAS

In certain embodiments, miRNA agents are targeted to specific cells or tissues. A targeted intracellular delivery of miRNAs to specific cells or tissues is desirable for enhancing the safety and efficacy of RNA interference-based therapeutics. Aptamers are oligonucleic acid or peptide molecules that bind to a wide range of specific target molecules with high specificity and affinity. High-affinity aptamers that target various protein families including cytokines, proteases, kinases, cell-surface receptors and cell-adhesion molecules have been synthesized. Aptamers are excellent tools for chemical biology, research, diagnosis, therapeutic delivery and monitoring therapy in real-time imaging. These properties together with their small size and their ease of large-scale synthesis make aptamers promising for targeted therapeutic applications. The approval of an anti-vascular endothelial growth factor (VEGF) aptamer (Eyetech/Pfizer's Macugen) in 2004 by the Food and Drug Administration (FDA) for treatment of age-related human macular degeneration has validated the therapeutic utility of the aptamer technology.

Nucleic acid-based aptamers targeting cell surface molecules are attractive delivery vehicles of various cargos to treat a distinct disease in a cell-type specific manner. Aptamers are 20-80 base pair long single-stranded deoxyribonucleic acid (DNA) or ribonucleic acid (RNA) oligonucleotides, which are folded into unique three-dimensional conformations due to various intra-molecular interactions. Aptamers allow delivery of molecules (e.g. siRNAs) that are not otherwise taken up efficiently by cells. RNA aptamers have been selected for various therapeutic targets including lysozyme, thrombin, factor IXa, human immunodeficiency virus trans-acting responsive element (HIV TAR), hemin, interferon gamma, vascular endothelial growth factor and dopamine. Aptamers offer several advantages over antibodies as they can be engineered completely in a test tube, are readily produced by straightforward chemical synthesis, possess desirable storage properties, and elicit little or no immunogenicity in therapeutic applications.

Chimeric aptamer approaches (where one molecule binds to the target and the other has a functional effect on the target molecule or cell) have generated more stable and efficient chimeric aptamers. Several chimerization strategies are available, including aptamer-aptamer, aptamer-nonaptamer biomacromolecules (siRNAs, proteins) and aptamer-nanoparticle chimeras. These chimeric aptamers when conjugated with various biomacromolecules like locked nucleic acid (LNA) to potentiate their stability, biodistribution, and targeting efficiency, have facilitated accurate targeting in preclinical trials.

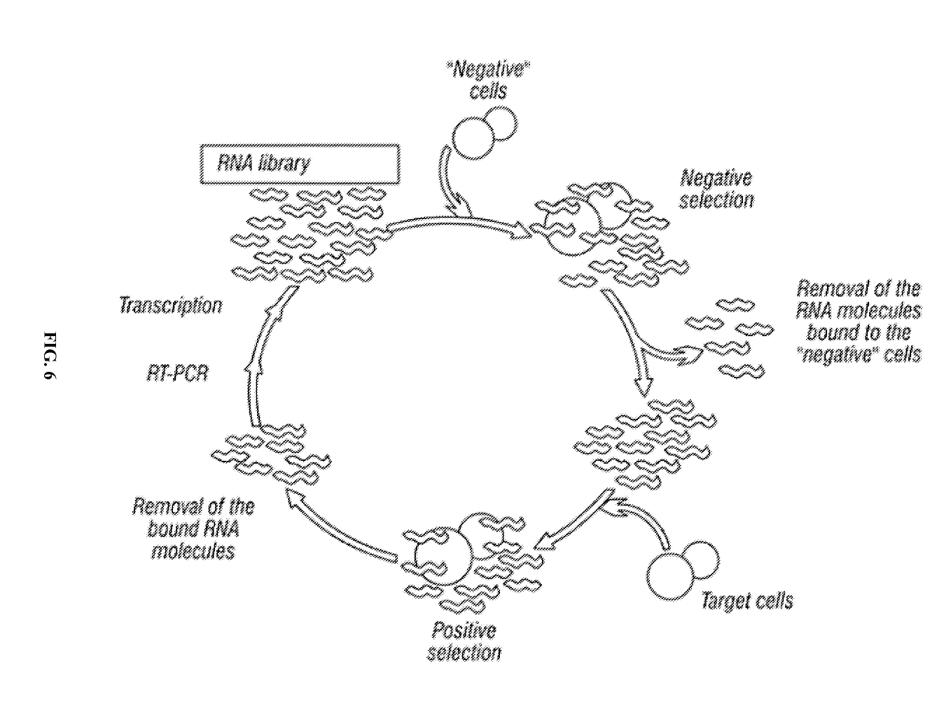

Given a specific molecular target, aptamers can be identified from combinatorial libraries of nucleic acids by a technique called Systematic Evolution of Ligands by EXponential Enrichment (SELEX) (See FIG. 6).

Currently, a number of aptamers targeting specific cell surface receptors have been successfully adapted for the selective delivery of active drug substances both in vitro and in vivo, including anti-cancer drugs, toxins, enzymes, radionuclides, virus, and siRNAs. A prostate specific membrane antigen aptamer has been used to deliver a siRNA targeting P1K1 for the treatment of prostate cancer. An RNA aptamer (with high binding affinity to the HIV-1 envelope gp120 protein and virus neutralization properties) attached to and delivering a small interfering RNA (siRNA) triggers sequence-specific degradation of HIV RNAs and protects from helper CD4+ T cell decline in humanized mice. The small size of nanoparticles allows them to interact readily with biomolecules both on surface and within the cells and thus provide targeted delivery of miRNAs to specific cells.

### C. SELECTION OF THE TARGETED CELLS

Selective delivery of miRNA analogs for the treatment of chronic visceral inflammation requires targeting specific cell types, such as pre-adipocyte, adipocyte, adipose tissue derived mesenchymal stem cell (ATMSC), stroma vascular cell, macrophage, lymphocyte, endothelial cell, vascular cell, fibroblast, hepatocyte and myocyte. In order to achieve therapeutic success, several cell types may be targeted.

1. Selection of Aptamers Based on known Targeted Cell Surface Molecules

a. AT-MSCs

Adipose tissue-derived mesenchymal stem cells (AT-MSCs) can differentiate into multiple lineages such as adipocytes, osteocytes, and chondrocytes, a property of therapeutic value. ATMSCs are present in human subcutaneous adipose tissue in appreciable quantities. From 1 g of adipose tissue, 5,000 stem cells can be isolated, which is 500 times more cells than from an equivalent amount of bone marrow. Human ATMSCs can be reprogrammed to become brown adipocytes (BAT) via modulation of a defined set of transcription factors.

Several ATMSC surface markers (CD9 (tetraspan), CD10 (MME), CD13 (ANPEP), CD29 (β-1 integrin), CD36 (FAT), CD44 (hyaluronate), CD49d (α-4 integrin), CD54 (ICAM-1), CD55 (DAF), CD59, CD73 (NT5E), CD90 (Thy1), CD91 (LPR1), CD105 (SH2, Endoglin), CD137, CD146 (Muc 18), CD166 (ALCAM), and HLA-ABC) can be targeted by aptamers, thus enhancing the selective delivery to these cells of miRNA agents for modulating inflammation. ATMSCs can be further selected by the lack of hematopoietic lineage markers such as CD11b, CD14, CD18, CD19, CD31, CD34, CD45, CD79 alpha, c-kit, STRO-1 and HLA-DR. Knowledge of these ATMSCs positive and negative cell surface markers allows their isolation by Flow Cytometry Cell Sorting (FACS) for subsequent screening and selection of aptamers by SELEX and Cell-SELEX technology.

b. WAT

White adipocytes (WAT) are the source of many adipocytokines involved in the adipocyte-macrophage paracrine loops and visceral inflammation. They can also be reprogrammed to become BAT (so-called brite or beige adipocytes) or to alter their lipid synthesis, storage and degradation functions. Thus, several cell surface markers of adipocytes were selected to allow their isolation by FACS and subsequent screening and selection of aptamers by SELEX and Cell-SELEX technology. Exemplary adipocyte cellular markers include caveolin-1 (CAV1), caveolin-2 (CAV2), CD10 (MME), CD36 (FAT), CD90 (Thy-1), CD91 (low density lipoprotein receptor-related protein 1, LRP1), CD140A (platelet-derived growth factor receptor, alpha polypeptide, PDGFRA), CD140B (platelet-derived growth factor receptor, alpha polypeptide, PDGFRB), CD146 (cell surface glycoprotein MUC18, MCAM), CD166 (activated leukocyte cell adhesion molecule, ALCAM), CLTCL1 (clathrin heavy chain-like 1), DCN (decorin), DPT (dermatopontin), FABP4 (fatty acid binding protein 4), LAMP1 (lysosomal-associated membrane protein 1), LAMP2 (lysosomal-associated membrane protein 2), NPR1 (Natriuretic peptide receptor A), SLC2A4 (glucose transporter 4, GLUT4), SLC27A1 (FATP1), SLC27A2 (FATP2) and SLC27A3 (FATP3).

c. Macrophages

Macrophages are the source of many pro-inflammatory and anti-inflammatory molecules (see e.g., FIG. 5). They actively communicate with other cell types and as such represent an obvious target for the treatment of chronic visceral inflammation. Thus, several cell surface markers of macrophages were selected to allow their isolation by FACS and subsequent screening and selection of aptamers by SELEX and Cell-SELEX technology. Furthermore, distinction is made between cellular markers of M1 pro-inflammatory macrophages and those of M2 anti-inflammatory macrophages. Exemplary M1 Macrophage cell surface markers include CCR5 (CD195), CCR7 (CD197), CD14, CD33, CD40 (TNFRSF5), CD68, CD80, CD86, CSF1R (CD115), EMR1 (Egf-like module containing, mucin-like, hormone receptor-like 1), Eng (CD105, Endoglin), FCGR1A (CD64), FCGR2A (CD32), FCGR2B (CD32), FCGR3A (CD16a), FCGR3B (CD16b), FUT4 (CD15), IL15RA (CD215), IL1R1 (CD121a), ITGAL (CD11a, Integrin alpha L), ITGAM (CD11b, Integrin alpha M, Mac-1), ITGAX (CD11c, Integrin alpha X), Lamp2 (CD107b, Mac3), LGALS3 (MAC2), PTPRC (CD45), SLAMF7 (CD319), TLR2 (CD282), TLR4 (CD284) and TNFRSF1B (CD120b). Exemplary M2 Macrophage cell surface markers include CD1A, CD1B, CD163, CD68, CD93, CD209, CD226, CD302 (DCL-1), CLEC4A (Dectin-1), FCER2 (CD23), IL1R2 (CD121b), IL27RA and MRC1 (CD206).

d. Hepatocytes

Non-alcoholic fatty liver disease (NAFLD) and non-alcoholic steatohepatitis (NASH) are often associated with chronic visceral inflammation. Thus, several cell surface markers of hepatocytes were selected to allow their isolation by FACS and subsequent screening and selection of aptamers by SELEX and Cell-SELEX technology. Exemplary hepatocyte cell surface markers include ABCG5 (ATP-binding cassette sub-family G member 5), ABCG8 (ATP-binding cassette sub-family G member 8), ASGP-R1 (Asialoglycoprotein-receptor 1), CD13, CD81, MDR-1 (P-glycoprotein 1, ABCB1), SLC27A5 (FATP5), SLCO1B1 (Solute carrier organic anion transporter family member 1B1), SLCO1B3 (Solute carrier organic anion transporter family member 1B3) and SR-B1 (Scavenger receptor class B member 1).

e. Vascular Endothelial Cells

Vascular Endothelial cells are damaged in cardiovascular and metabolic diseases. Preservation of their integrity ought to prevent these disorders. Thus, several cell surface markers of vascular endothelial cells were selected to allow their isolation by FACS and subsequent screening and selection of aptamers by CELEX and Cell-SELEX technology. Exemplary vascular endothelial cell surface markers include CD143/ACE, COLEC11/CL-K1, EPOR/erythropoietin receptor, ITGB7, LYVE1, E-Selectin/CD62E, TEK/CD2028, FLT4/VEGFR3, CD93/C1QR1, CL-P1/COLEC12, ESAM, ITGB2/CD18, CD146/MCAM, SELE/E-Selectin (CD62E), TNFRSF1A/CD120a, AGGF1/VG5Q, CDH5/VE-Cadherin, SELP/P-Selectin (CD62P), FABP5/E-FABP, KLF4, CD112/PVRL2, F3/Coagulation Factor III/CD142/Tissue Factor, TNFRSF1B/CD120b, VWA2, ACKR2/atypical Chemokine Receptor 2, CLEC4M/CD299, FABP6, TYMP/Thymidine Phosphorylase, SLAMF1/CD150, TRA 1-85, CD31/PECAM1, DCBLD2/ESDN, ICAM-1/CD54, PDPN/Podocalyxin, STAB1/Stabilin 1, TNFRSF10A/CD261, CD34, BSG/EMMPRIN, ICAM-2/CD102, PDPN/Podoplanin, STAB2/Stabilin 2, TNFRSF10B/CD262, CD36/FAT, Endoglin/CD105, IL1R1/CD121A, S1PR1/EDG1, PLXDC1/TEM7, Vcam1/CD106, CD151, EMCN/Endomucin, IL13RA1/CD213A1, 51PR2/EDG5, ANTXR1/TEM8, EGFL7/VE-STATIN, CD160, CD248/Endosialin, ITGA4/Integrin alpha 4/CD49D, S1PR3/EDG3, THBD/Thrombomodulin/CD141, FLT1/VEGFR1, CD300LG/Nepmucin, PROCR/CCD41, ITGB1/CD29, S1PR5/EDG8, THSD1/Thrombospondin, type 1, KDR/VEGFR2/CD309.

2. Selection of Aptamers based on Target Cells

Cell-SELEX does not require knowledge and availability of isolated purified cell surface markers. Cell-SELEX relies on more physiological conditions when the protein is displayed on the living intact cell surface. Therefore the Cell-SELEX approach is used with the specific cell types mentioned above, according to a selection process that does not require a priori knowledge and/or availability of specific surface markers for that specific cell type.

### D. CROSS TALK BETWEEN CELLS

Secreted vesicles constitute a heterogeneous population of vesicles with a diameter from 30 to 100 nm (exosomes) and from 100 to 1000 nm (microparticles, MPs). Secreted vesicles are released by budding of the cellular plasma membrane and express antigens that are specific of their parental cells. Exosomes and MPs display specific surface receptors and carry a broad spectrum of bioactive substances (cytokines, signaling proteins, mRNAs, and microRNAs). Exosomes and MPs function as vectors for the intercellular exchange of biological signals and information, leading to cell activation, phenotypic modification, and reprogramming of cell functions. While exosomes and MPs formation and release represent a physiological phenomenon, a significant increase in circulating exosomes and MPs is observed in various pathologies, including inflammatory and autoimmune disorders, cardiovascular and metabolic diseases, atherosclerosis, and malignancies. In the context of chronic inflammation, paracrine loops involving free fatty acids (FFA) and TNF-alpha between adipocytes and macrophages establish a vicious cycle that aggravates inflammatory changes in the adipose tissue. Similarly, human perivascular WAT (pWAT) can secrete different chemokines and might contribute to the progression of obesity-associated atherosclerosis

### E. METHODS OF TREATMENT

In one aspect, the disclosure provides a method of modulating the inflammatory activity of a cell associated with chronic visceral inflammation. In another aspect, the disclosure provides a method of treating visceral inflammation in a human subject. The method general comprises administering to the human subject an effective amount of a miRNA agent that modulates activity of at least one inflammation regulator or modulator.

“Treatment”, or “treating” as used herein, is defined as the application or administration of a therapeutic agent (e.g., a miRNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

Such methods of treatment may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

miRNA agents can be tested in an appropriate animal model e.g., an obesity model with insulin resistance and visceral inflammation. For example, a miRNA agent (or expression vector or transgene encoding same) as described herein can be used in an animal model to determine the efficacy, toxicity, or side effects of treatment with said agent. Alternatively, a therapeutic agent can be used in an animal model to determine the mechanism of action of such an agent. For example, a miRNA agent can be used in an animal model to determine the efficacy, toxicity, or side effects of treatment with such an agent. Alternatively, an agent can be used in an animal model to determine the mechanism of action of such an agent.

A miRNA agent modified for enhance uptake into cells (e.g., adipose cells) can be administered at a unit dose less than about 15 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of miRNA agent (e.g., about 4.4.times.10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA silencing agent per kg of bodyweight. The unit dose, for example, can be administered by injection (e.g., intravenous or intramuscular), an inhaled dose, or a topical application. Particularly preferred dosages are less than 2, 1, or 0.1 mg/kg of body weight.

Delivery of an miRNA agent directly to an organ or tissue (e.g., directly to adipose or liver tissue) can be at a dosage on the order of about 0.00001 mg to about 3 mg per organ/tissue, or preferably about 0.0001-0.001 mg per organ/tissue, about 0.03-3.0 mg per organ/tissue, about 0.1-3.0 mg per organ/tissue or about 0.3-3.0 mg per organ/tissue. The dosage can be an amount effective to treat or prevent obesity. In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. In one embodiment, the effective dose is administered with other traditional therapeutic modalities.

In certain embodiment, a subject is administered an initial dose, and one or more maintenance doses of a miRNA agent. The maintenance dose or doses are generally lower than the initial dose, e.g., one-half less of the initial dose. A maintenance regimen can include treating the subject with a dose or doses ranging from 0.01 mg/kg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time, which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in condition, e.g., changes in percentage body fat. The dosage of the compound may either be increased in the event the patient does not respond significantly to current dosage levels, or the dose may be decreased if for instance a decrease in body fat is observed, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., sub-cutaneous, intravenous, intraperitoneal, intracisternal or intracapsular), or reservoir may be advisable. In one embodiment, a pharmaceutical composition includes a plurality of miRNA agent species. In another embodiment, the miRNA agent species has sequences that are non-overlapping and non-adjacent to another species with respect to a naturally occurring target sequence. In another embodiment, the plurality of miRNA agent species is specific for different naturally occurring target genes. In another embodiment, the miRNA agent is allele specific. In another embodiment, the plurality of miRNA agent species target two or more SNP alleles (e.g., two, three, four, five, six, or more SNP alleles).

Following successful treatment, it may be desirable to have the patient undergo maintenance therapy to prevent the recurrence of the disease state, wherein the compound of the invention is administered in maintenance doses, ranging from 0.01 mg per kg to 100 mg per kg of body weight (see U.S. Pat. No. 6,107,094).

The “effective amount” of the miRNA agent composition is an amount sufficient to be effective in treating or preventing a disorder or to regulate a physiological condition in humans. The concentration or amount of miRNA agent administered will depend on the parameters determined for the agent and the method of administration, e.g. nasal, buccal, or pulmonary. For example, nasal formulations tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a miRNA agent can include a single treatment or, preferably, can include a series of treatments. It will also be appreciated that the effective dosage of a miRNA agent for treatment may increase or decrease over the course of a particular treatment. Changes in dosage may result and become apparent from the results of diagnostic assays as described herein. For example, the subject can be monitored after administering a miRNA agent composition. Based on information from the monitoring, an additional amount of the miRNA agent composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human gene, e.g., a gene that produces a target mRNA (e.g., a inflammation regulator). The transgenic animal can be deficient for the corresponding endogenous mRNA. In another embodiment, the composition for testing includes a miRNA agent that is complementary, at least in an internal region, to a sequence that is conserved between a nucleic acid sequence in the animal model and the target nucleic acid sequence in a human.

miRNA agents may be directly introduced into a cell (e.g., an adipocyte, an hepatocyte, a macrophage, a lymphocyte, a myocyte); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The miRNA agents of the invention can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The miRNA agents may be introduced along with other components e.g., compounds that enhance miRNA agent uptake by a cell.

### F. SCREENING METHODS

In another aspect, the invention provides, a method of screening for a miRNA agent that modulates inflammation. The method generally comprises the steps of: providing an indicator cell comprising a human genome; contacting the indicator cell with a test miRNA agent; and determining the cellular activity of at least one inflammation regulator in the indicator cell in the presence and absence of the miRNA agent, wherein a change in the activity of the inflammation regulator in the presence of the test miRNA agent identifies the test miRNA agent as a miRNA agent that modulates inflammation.

Any inflammation regulator can be assayed in the methods disclosed herein. Exemplary inflammation regulators include AICDA, AIM2, AKT2, ANGPTL2 (angiopoietin-like 2), CASP1 (Caspase 1), CCL2 (Chemokine C-C motif ligand 2, MCP-1), CCL3 (MIP-1 alpha), CCL4 (MIP-1 beta), CCL5 (RANTES), CCL7 (MCP-3), CCL8 (MCP-2), CCL11 (Eotaxin), CCL15 (MIP-1 delta), CCL17 (TARC), CCL19 (MIP-3b), CCL20 (MIP-3-alpha), CCL22 (MDC), CCR2 (Chemokine C-C motif receptor 2, MCP-1-R), CCR7 (CD197), CD40LG (CD154), CD69, CEBPA, CEBPB, CFD (Adipsin), CHUK (IKKA), CXCR4 (Chemokine C-X-C motif receptor 4, CD 184), MT-CO2 (Cytochrome c oxidase subunit II), CRP, CXCL1, CXCL2, CXCL3, CXCL5, CXCL9 (MIG), CXCL10 (IP-10), CXCL11 (ITAC), CXCL12 (SDF-1), CXCL13 (BCA1), CXCL16, FGA (Fibrinogen A), FGB (Fibrinogen B), FGG (Fibrinogen G), HIF1A, ICAM1 (CD54), IFNB1, IFNG, IKBKB (IKKB), IKBKE (IKKE), IL12A, IL12B, IL15, IL17A, IL18, IL1A, IL1B, IL2, IL23A, IL6, IL8 (CXCL8), INPP5D, IRAK1, IRAK2, IRF3 (Interferon-regulatory factor 3), IRF4 (Interferon-regulatory factor 4), IRF5 (Interferon-regulatory factor 5), LCN2 (Lipocalin 2), LEP (Leptin), MAP3K7, MMP2, MMP9, MYD88, NAMPT (Visfatin), NFKB1, NLRP3 (NLR family, pyrin domain containing 3), NOS2 (iNOS), SPP1 (Osteopontin), P2RX7 (Purinergic receptor P2X7), PDCD4, PKNOX1, PTX3 (Pentraxin 3), RBP4, RETN (Resistin), SAA1 (serum amyloid A1), SELE (E-selectin), SELP (P-selectin), SERPINE1 (plasminogen activator inhibitor type 1), SOCS1, SOCS3, SPI1, STAT1, TAB1, TAB2, TGFB1, TIRAP, TLR2 (CD282), TLR3 (CD283), TLR4 (CD284), TLR6 (CD286), TLR7, TLR8 (CD288), TNF (TNF alpha), TRAF6, TXNIP (Thioredoxin interacting protein), VCAM1, VEGFA, WNT5A, ADIPOQ (Adiponectin), AKT1, ALOX15, ARG1 (Arginase 1), ARG2 (Arginase 2), CCL16, CCL18, CCL24, CD36, CTBP1, CXCR1, CXCR2, FABP4, HDAC3, IL10, IL10RA (IL10R), IL13, IL1RN (IL1RA), IL3, IL33, IL4, IL4R, IL5, KDM6B (JMJD3), KLF2, KLF4, MAF (c-MAF), MRC1 (Macrophage mannose receptor 1), MT-COX3, MYC (c-Myc), NR1H3 (LXRA), PPARD, PPARG, PPARGC1A, PRDX3 (Peroxiredoxin), RETNLB (FIZZ1), SCARB1, SFRP5, SIRT1, SIRT2, SIRT3, SIRT4, SIRT5, SIRT6, SIRT7, SOCS2, STAT3, and STATE.

Any cell in which the activity of an inflammation regulator can be measured is suitable for use in the methods disclosed herein. Exemplary cells include adipocytes, adipose tissue derived mesenchymal stem cells, hepatocytes, myocytes, or precursors thereof. Any activity of an inflammation regulator can be assayed, including, without limitation, mRNA expression level, protein expression level or activity of the inflammation regulator. Methods for determining such activities are well known in the art. Any miRNA agent can be screened, including, without limitation, miRNA, agomirs, antagomirs, aptamirs, miR-masks, miRNA sponges, siRNA (single- or double-stranded), shRNA, antisense oligonucleotides, ribozymes, or other oligonucleotide mimetics, which hybridize to at least a portion of a target nucleic acid and modulate its function.

### G. PHARMACEUTICAL COMPOSITIONS

In one aspect, the methods disclosed herein can include the administration of pharmaceutical compositions and formulations comprising miRNA agents capable of modulating the activity of at least one inflammation regulator or modulator.

In certain embodiments, the compositions are formulated with a pharmaceutically acceptable carrier. The pharmaceutical compositions and formulations can be administered parenterally, topically, by direct administration into the gastrointestinal tract (e.g., orally or rectally), or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy. 21st ed., 2005.

The miRNA agents can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations of the invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients, which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragées, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragée cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In certain embodiments, oil-based pharmaceuticals are used for administration of the miRNA agents. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, 1997.

In certain embodiments, the pharmaceutical compositions and formulations are in the form of oil-in-water emulsions. The oily phase can be a vegetable oil or a mineral oil, described above, or a mixture of these. Suitable emulsifying agents include naturally occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise paraffin oil, a sorbitan monooleate, an ethoxylated sorbitan monooleate and/or an ethoxylated sorbitan trioleate.

In certain embodiments, the pharmaceutical compositions and formulations are administered by in intranasal, intraocular and intravaginal routes including suppositories, insufflation, powders and aerosol formulations (for examples of steroid inhalants, see e.g., Rohatagi, 1995; Tjwa, 1995. Suppositories formulations can be prepared by mixing the drug with a suitable non-irritating excipient, which is solid at ordinary temperatures but liquid at body temperatures and will therefore melt in the body to release the drug. Such materials are cocoa butter and polyethylene glycols.

In certain embodiments, the pharmaceutical compositions and formulations are delivered transdermally, by a topical route, formulated as applicator sticks, solutions, suspensions, emulsions, gels, creams, ointments, pastes, jellies, paints, powders, and aerosols.

In certain embodiments, the pharmaceutical compositions and formulations are delivered as microspheres for slow release in the body. For example, microspheres can be administered via intradermal injection of drug, which slowly release subcutaneously; see Rao, 1995; as biodegradable and injectable gel formulations, see, e.g., Gao, 1995; or, as microspheres for oral administration, see, e.g., Eyles, 1997.

In certain embodiments, the pharmaceutical compositions and formulations are parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well-known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In certain embodiments, the pharmaceutical compounds and formulations are lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

In certain embodiments, the pharmaceutical compositions and formulations are delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed, 1996; Chonn, 1995; Ostro, 1989.

The formulations of the invention can be administered for prophylactic and/or therapeutic treatments. In certain embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in certain embodiments, pharmaceutical compositions of the invention are administered in an amount sufficient to treat obesity in a subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones, 1996; Groning, 1996; Fotherby, 1996; Johnson, 1995; Rohatagi, 1995; Brophy, 1983; Remington: The Science and Practice of Pharmacy, 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate. Single or multiple administrations of formulations can be given depending on for example: the dosage and frequency as required and tolerated by the patient, the degree and amount of cholesterol homeostasis generated after each administration, and the like. The formulations should provide a sufficient quantity of active agent to effectively treat, prevent or ameliorate conditions, diseases or symptoms, e.g., treat obesity.

In certain embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 2005.

Several studies have reported successful mammalian dosing using miRNA agents. For example, Esau, et al, 2006 reported dosing of normal mice with intraperitoneal doses of miR-122 antisense oligonucleotide ranging from 12.5 to 75 mg kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, nontoxic dose. Another study by Krutzfeldt, et al, 2005, injected antagomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen, et al, 2008, report that efficient silencing of miR-122 was achieved in primates by three doses of 10 mg per kg LNA-antimiR, leading to a long-lasting and reversible decrease in total plasma cholesterol without any evidence for LNA-associated toxicities or histopathological changes in the study animals.

In certain embodiments, the methods described herein include co-administration of miRNA agents with other drugs or pharmaceuticals, e.g., compositions for modulating inflammation.

### H. EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

Furthermore, in accordance with the present invention there may be employed conventional molecular biology, microbiology, and recombinant DNA techniques within the skill of the art. Such techniques are explained fully in the literature. See, e.g., Sambrook, Fritsch & Maniatis, 1989; Glover, 1985; Gait, 1984; Hames & Higgins, 1985; Hames & Higgins, 1984; Freshney, 1986; Immobilized Cells And Enzymes, 1986; Perbal, 1984; Ausubel, et al., 1994.

### Example 1

### In Silico Systems Biology Reconstruction of Chronic Visceral Inflammation

Accurate physiological representation and analysis of systemic diseases require an integrated multilevel and comprehensive modeling approach relying upon an appropriate computational infrastructure (Systems Biology). Genome-scale metabolic network reconstructions have been shown to provide an appropriate context for analyzing biological content. A global human metabolic network, termed Recon 1, has recently been reconstructed allowing the systems analysis of human metabolic physiology and pathology. Utilizing high throughput data, Recon 1 was tailored to different cells and tissues, including the liver, kidney, brain, and alveolar macrophage. Recon 1 was consequently used to describe metabolism in three human cells: adipocytes, hepatocytes, and myocytes. This novel multi-tissue type modeling approach was developed to integrate the metabolic functions for the three cell types, and subsequently used to simulate known integrated metabolic cycles. In addition, the multi-tissue model was used to study diabetes, a pathology with systemic properties. High-throughput data was integrated with the network to determine differential metabolic activity between obese and type 2 DM obese gastric bypass patients in a whole-body context. Such Systems Biology models were successfully used to construct a genome-scale metabolic network for the RAW 264.7 macrophage cell line to determine metabolic modulators of macrophage activation and to construct a cell-specific alveolar macrophage model, iAB-AMO-1410 which successfully predicted experimentally verified ATP and nitric oxide production rates in macrophages. Several publications have utilized Recon 1 to study cancer, diabetes, host-pathogen interactions, heritable metabolic disorders and off-target drug binding effects.

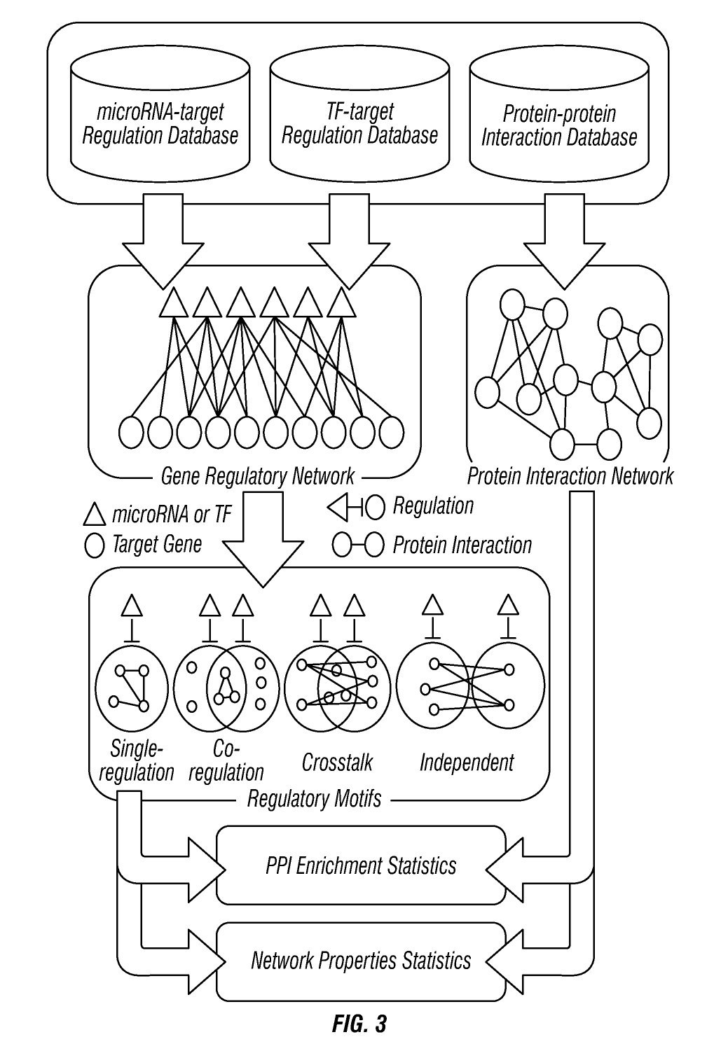

Gene regulatory networks (GRNs) are defined as comprehensive collections of regulatory relationships that control the global gene expression and the dynamics of protein output in a living cell (see FIG. 3). Integrated GRNs are used to decipher regulatory relationships at the transcriptional and post-transcriptional levels. Four patterns of interactions between transcription factors and miRNAs (key elements of GRNs) and proteins have been described: single-regulation, co-regulation, crosstalk, and independent. The crosstalk pattern has been found to have the most enriched protein-protein interactions in their downstream regulatory targets.

A Systems Biology integrated multilevel and comprehensive modeling approach is used to reconstruct an in silico GRN model of visceral inflammation in human that includes adipose tissue, macrophages, lymphocytes, vascular wall, hepatocytes, and myocytes. This model is built in a “Learn and Confirm” fashion, whereby experimental data from in vitro and in vivo experiments are included to calibrate, validate and refine the model.

### Example 2

### In Silico Analysis of Inflammation Regulators

One hundred and fifty three molecules that are involved in inflammation were selected based upon a critical assessment and review of the available scientific information. These molecules were categorized as pro- and anti-inflammatory molecules based upon their functions. These inflammation regulators are set forth in Table 1, herein.

TABLE 1
Exemplary Inflammation Regulators

| | | Entrez # | Gene # |
|---|---|---|---|
| | Pro-inflammatory Molecules | | |
| 1 | AICDA | 57379 | ENSG00000111732 |
| 2 | AIM2 | 9447 | ENSG00000163568 |
| 3 | AKT2 | 208 | ENSG00000105221 |
| 4 | ANGPTL2 (angiopoietin-like 2) | 23452 | ENSG00000136859 |
| 5 | CASP1 (Caspase 1) | 834 | ENSG00000137752 |
| 6 | CCL2 (Chemokine C-C motif | 6347 | ENSG00000108691 |
| | ligand 2, MCP-1) | | |
| 7 | CCL3 (MIP-1 alpha) | 6348 | ENSG00000006075 |
| 8 | CCL4 (MIP-1 beta) | 6351 | ENSG00000129277 |
| 9 | CCL5 (RANTES) | 6352 | ENSG00000161570 |
| 10 | CCL7 (MCP-3) | 6354 | ENSG00000108688 |
| 11 | CCL8 (MCP-2) | 6355 | ENSG00000108700 |
| 12 | CCL11 (Eotaxin) | 6356 | ENSG00000172156 |
| 13 | CCL15 (MIP-1 delta) | 6359 | ENSG00000161574 |
| 14 | CCL17 (TARC) | 6361 | ENSG00000102970 |
| 15 | CCL19 (MIP-3b) | 6363 | ENSG00000172724 |
| 16 | CCL20 (MIP-3-alpha) | 6364 | ENSG00000115009 |
| 17 | CCL22 (MDC) | 6367 | ENSG00000102962 |
| 18 | CCR2 (Chemokine C-C motif | 729230 | ENSG00000121807 |
| | receptor 2, MCP-1-R) | | |
| 19 | CCR7 (CD197) | 1236 | ENSG00000126353 |
| 20 | CD40LG (CD154) | 959 | ENSG00000102245 |
| 21 | CD69 | 969 | ENSG00000110848 |
| 22 | CEBPA | 1050 | ENSG00000245848 |
| 23 | CEBPB | 1051 | ENSG00000172216 |
| 24 | CFD (Adipsin) | 1675 | ENSG00000197766 |
| 25 | CHUK (IKKA) | 1147 | ENSG00000213341 |
| 26 | CXCR4 (Chemokine C-X-C motif | 7852 | ENSG00000121966 |
| | receptor 4, CD 184) | | |
| 27 | MT-CO2 (Cytochrome c oxidase | 4513 | ENSG00000198712 |
| | subunit II) | | |
| 28 | CRP | 1401 | ENSG00000132693 |
| 29 | CXCL1 | 2919 | ENSG00000163739 |
| 30 | CXCL2 | 2920 | ENSG00000081041 |
| 31 | CXCL3 | 2921 | ENSG00000163734 |
| 32 | CXCL5 | 6374 | ENSG00000163735 |
| 33 | CXCL9 (MIG) | 4283 | ENSG00000138755 |
| 34 | CXCL10 (IP-10) | 3627 | ENSG00000169245 |
| 35 | CXCL11 (ITAC) | 6373 | ENSG00000169248 |
| 36 | CXCL12 (SDF-1) | 6387 | ENSG00000107562 |
| 37 | CXCL13 (BCA1) | 10563 | ENSG00000156234 |
| 38 | CXCL16 | 58191 | ENSG00000161921 |
| 39 | FGA (Fibrinogen A) | 2243 | ENSG00000171560 |
| 40 | FGB (Fibrinogen B) | 2244 | ENSG00000171564 |
| 41 | FGG (Fibrinogen G) | 2266 | ENSG00000171557 |
| 42 | HIF1A | 3091 | ENSG00000100644 |
| 43 | ICAM1 (CD54) | 3383 | ENSG00000090339 |
| 44 | IFNB1 | 3456 | ENSG00000171855 |
| 45 | IFNG | 3458 | ENSG00000111537 |
| 46 | IKBKB (IKKB) | 3551 | ENSG00000104365 |
| 47 | IKBKE (IKKE) | 9641 | ENSG00000143466 |
| 48 | IL12A | 3592 | ENSG00000168811 |
| 49 | IL12B | 3593 | ENSG00000113302 |
| 50 | IL15 | 3600 | ENSG00000164136 |
| 51 | IL17A | 3605 | ENSG00000112115 |
| 52 | IL18 | 3606 | ENSG00000150782 |
| 53 | IL1A | 3552 | ENSG00000115008 |
| 54 | IL1B | 3553 | ENSG00000125538 |
| 55 | IL2 | 3558 | ENSG00000109471 |
| 56 | IL23A | 51561 | ENSG00000110944 |
| 57 | IL6 | 3569 | ENSG00000136244 |
| 58 | IL8 (CXCL8) | 3576 | ENSG00000169429 |
| 59 | INPP5D | 3635 | ENSG00000168918 |
| 60 | IRAK1 | 3654 | ENSG00000184216 |
| 61 | IRAK2 | 3656 | ENSG00000134070 |
| 62 | IRF3 (Interferon-regulatory | 3661 | ENSG00000126456 |
| | factor 3) | | |
| 63 | IRF4 (Interferon-regulatory | 3662 | ENSG00000137265 |
| | factor 4) | | |
| 64 | IRF5 (Interferon-regulatory | 3663 | ENSG00000128604 |
| | factor 5) | | |
| 65 | LCN2 (Lipocalin 2) | 3934 | ENSG00000148346 |
| 66 | LEP (Leptin) | 3952 | ENSG00000174697 |
| 67 | MAP3K7 | 6885 | ENSG00000135341 |
| 68 | MMP2 | 4313 | ENSG00000087245 |
| 69 | MMP9 | 4318 | ENSG00000100985 |
| 70 | MYD88 | 4615 | ENSG00000172936 |
| 71 | NAMPT (Visfatin) | 10135 | ENSG00000105835 |
| 72 | NFKB1 | 4790 | ENSG00000109320 |
| 73 | NLRP3 (NLR family, pyrin | 114548 | ENSG00000162711 |
| | domain containing 3) | | |
| 74 | NOS2 (iNOS) | 4843 | ENSG00000007171 |
| 75 | SPP1 (Osteopontin) | 6696 | ENSG00000118785 |
| 76 | P2RX7 (Purinergic receptor P2X7) | 5027 | ENSG00000089041 |
| 77 | PDCD4 | 27250 | ENSG00000150593 |
| 78 | PKNOX1 | 5316 | ENSG00000160199 |
| 79 | PTX3 (Pentraxin 3) | 5806 | ENSG00000163661 |
| 80 | RBP4 | 5950 | ENSG00000138207 |
| 81 | RETN (Resistin) | 56729 | ENSG00000104918 |
| 82 | SAA1 (serum amyloid A1) | 6288 | ENSG00000173432 |
| 83 | SELE (E-selectin) | 6401 | ENSG00000007908 |
| 84 | SELP (P-selectin) | 6403 | ENSG00000174175 |
| 85 | SERPINE1 (plasminogen activator | 5054 | ENSG00000106366 |
| | inhibitor type 1) | | |
| 86 | SOCS1 | 8651 | ENSG00000185338 |
| 87 | SOCS3 | 9021 | ENSG00000184557 |
| 88 | SPI1 | 6688 | ENSG00000066336 |
| 89 | STAT1 | 6772 | ENSG00000115415 |
| 90 | TAB1 | 10454 | ENSG00000100324 |
| 91 | TAB2 | 23118 | ENSG00000055208 |
| 92 | TGFB1 | 7040 | ENSG00000105329 |
| 93 | TIRAP | 114609 | ENSG00000150455 |
| 94 | TLR2 (CD282) | 7097 | ENSG00000137462 |
| 95 | TLR3 (CD283) | 7098 | ENSG00000164342 |
| 96 | TLR4 (CD284) | 7099 | ENSG00000136869 |
| 97 | TLR6 (CD286) | 10333 | ENSG00000174130 |
| 98 | TLR7 | 51284 | ENSG00000196664 |
| 99 | TLR8 (CD288) | 51311 | ENSG00000101916 |
| 100 | TNF (TNF alpha) | 7124 | ENSG00000232810 |
| 101 | TRAF6 | 7189 | ENSG00000175104 |
| 102 | TXNIP (Thioredoxin | 10628 | ENSG00000117289 |
| | interacting protein) | | |
| 103 | VCAM1 | 7412 | ENSG00000162692 |
| 104 | VEGFA | 7422 | ENSG00000112715 |
| 105 | WNT5A | 7474 | ENSG00000114251 |
| | Anti-inflammatory Molecules | | |
| 1 | ADIPOQ (Adiponectin) | 9370 | ENSG00000181092 |
| 2 | AKT1 | 207 | ENSG00000142208 |
| 3 | ALOX15 | 246 | ENSG00000161905 |
| 4 | ARG1 (Arginase 1) | 383 | ENSG00000118520 |
| 5 | ARG2 (Arginase 2) | 384 | ENSG00000081181 |
| 6 | CCL16 | 6360 | ENSG00000161573 |
| 7 | CCL18 | 6362 | ENSG00000006074 |
| 8 | CCL24 | 6369 | ENSG00000106178 |
| 9 | CD36 | 948 | ENSG00000135218 |
| 10 | CTBP1 | 1487 | ENSG00000159692 |
| 11 | CXCR1 | 3577 | ENSG00000163464 |
| 12 | CXCR2 | 3579 | ENSG00000180871 |
| 13 | FABP4 | 2167 | ENSG00000170323 |
| 14 | HDAC3 | 8841 | ENSG00000171720 |
| 15 | IL10 | 3586 | ENSG00000136634 |
| 20 | IL10RA (IL10R) | 3587 | ENSG00000110324 |
| 21 | IL13 | 3596 | ENSG00000169194 |
| 22 | IL1RN (IL1RA) | 3557 | ENSG00000136689 |
| 16 | IL3 | 3562 | ENSG00000164399 |
| 17 | IL33 | 90865 | ENSG00000137033 |
| 18 | IL4 | 3565 | ENSG00000113520 |
| 19 | IL4R | 3566 | ENSG00000077238 |
| 23 | IL5 | 3567 | ENSG00000113525 |
| 24 | KDM6B (JMJD3) | 23135 | ENSG00000132510 |
| 25 | KLF2 | 10365 | ENSG00000127528 |
| 26 | KLF4 | 9314 | ENSG00000136826 |
| 27 | MAF (c-MAF) | 4094 | ENSG00000178573 |
| 28 | MRC1 (Macrophage mannose | 4360 | ENSG00000120586 |
| | receptor 1) | | |
| 29 | MT-COX3 | 4514 | ENSG00000198938 |
| 30 | MYC (c-Myc) | 4609 | ENSG00000136997 |
| 31 | NR1H3 (LXRA) | 10062 | ENSG00000025434 |
| 32 | PPARD | 5467 | ENSG00000112033 |
| 33 | PPARG | 5468 | ENSG00000132170 |
| 34 | PPARGC1A | 10891 | ENSG00000109819 |
| 35 | PRDX3 (Peroxiredoxin) | 10935 | ENSG00000165672 |
| 36 | RETNLB (FIZZ1) | 84666 | ENSG00000163515 |
| 37 | SCARB1 | 949 | ENSG00000073060 |
| 38 | SFRP5 | 6425 | ENSG00000120057 |
| 39 | SIRT1 | 23411 | ENSG00000096717 |
| 40 | SIRT2 | 22933 | ENSG00000068903 |
| 41 | SIRT3 | 23410 | ENSG00000142082 |
| 42 | SIRT4 | 23409 | ENSG00000089163 |
| 43 | SIRT5 | 23408 | ENSG00000124523 |
| 44 | SIRT6 | 51548 | ENSG00000077463 |
| 45 | SIRT7 | 51547 | ENSG00000187531 |
| 46 | SOCS2 | 8835 | ENSG00000120833 |
| 47 | STAT3 | 6774 | ENSG00000168610 |
| 48 | STAT6 | 6778 | ENSG00000166888 |

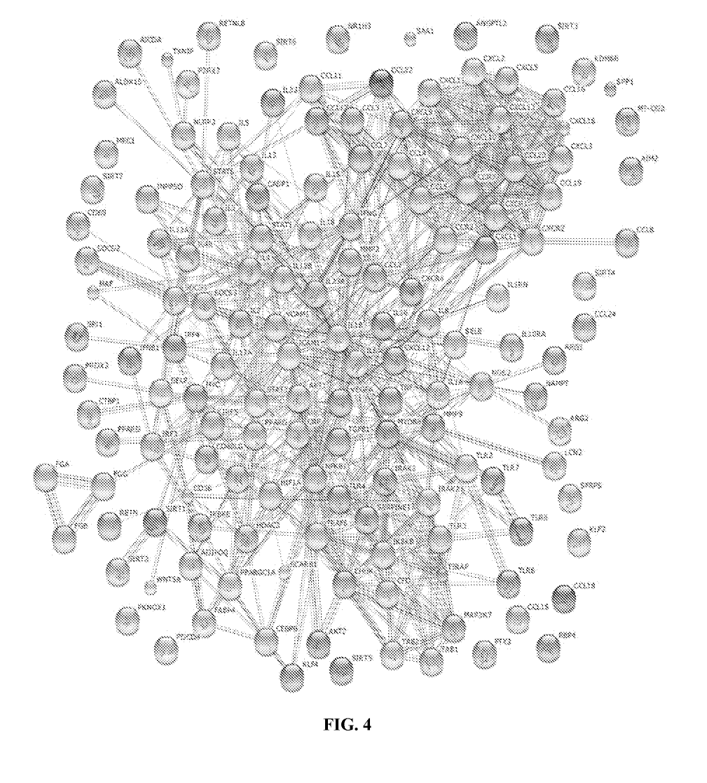

The STRING 9.05 database of known and predicted protein interactions (string-db.org) was used to explore these 153 candidate molecules. Their interactions include direct (physical) and indirect (functional) associations; they are derived from four sources: genomic context; high-throughput experiments; co-expression; and previous knowledge. STRING quantitatively integrates interaction data from these sources for a large number of organisms, and transfers information between these organisms where applicable. The database currently covers 5,214,234 proteins from 1,133 organisms. As an example, the relationships between these 153 inflammation regulators in Homo Sapiens are displayed in FIG. 4, using a high confidence score of 0.90. Such as a dense network of interactions among these many molecules suggests that the therapeutic use of miRNA analogs along the concept of one drug-several targets is quite appropriate in the indication of treating chronic inflammation.

### Example 3

### In Silico Selection of Relevant Mirna Targets

To select inflammation miRNA analogs, thirty-four internet-based resources were employed to match miRNAs and their targets (the “micronome”).

Specifically, these tools were used to perform: 1) Integrated Data Mining (8 tools); 2) miRNA Mining and Mapping (6 tools); 3) miRNA Target Targets and Expression (21 tools); 4) Integrated miRNA Targets and Expression (13 tools); 5) miRNA Secondary Structure Prediction and Comparison (5 tools); 6) Network Searches and Analyses (8 tools); 7) Molecular Visualization (4 tools); and 8) Information Integration and Exploitation (1 tool).

A single gene target can be controlled by several miRNAs whereas a single miRNA can control several gene targets. Sophisticated bioinformatics resources have been developed to select the most relevant miRNAs to target diseases (Gallagher, et al., 2010; Fujiki, et al., 2009; Okada, et al., 2010; Hao, et al., 2012; Hao, et al., 2012). However, the results of these algorithms are acutely dependent on predefined parameters and the degree of convergence between these algorithms is rather limited. Therefore, there is a need to develop better performing bioinformatics tools with improved sensitivity, specificity and selectivity for the identification of miRNA/target relationships.

Consequently, the inventors developed a system that scores pairs of miRNAs and genes and returns sorted lists of miRNAs for a given gene, and vice versa. This system also allows for a given set of genes (i.e. a gene network) to return a ranked list of miRNAs interacting with the network, based upon average score or rank of individual genes. Eight score matrices predicted by DianaMicroT, miRWalk, picTar4way, picTar5way, PITA, RNA22, TargetScan conserved and TargetScan non conserved, were created as well as four matrices of miRNA-gene interactions verified experimentally which were extracted from the following tools: Starbase, miRWalk, miRTarBase and miRRecords. These matrices have 19,061 rows (genes published in the HUGO gene nomenclature) and 2,043 columns (mature miRNAs published in miRBase version 19). The data were fused to form a single score matrix summarizing all such information, which is used for all ranking procedures. Queries can be either to rank all genes given a certain miRNA, or to rank all miRNAs given a certain gene. The data fusion method consists in first applying logistic regression to predict the All Verified Merged values from the 8 score values, then applying singular value decomposition and reconstruction of the score matrix to improve the resulting scores. Performance was assessed first by training using half of the verified values (complemented by an equal number of non verified values treated as negative outcomes). Validation testing on all the remaining values reached an ROC AUC of 0.91.

The inventors used a proprietary system to predict miRNAs from the InflaRNOme and the AdipoRNOme that potentially interact with the network of pro-inflammatory and anti-inflammatory molecules listed in table 3. The 44 miRNAs present in both the InflaRNOme and the AdipoRNOme, and predicted to interact with the pro-inflammatory molecules are: hsa-let-7a-5p, hsa-let-7b-5p, hsa-let-7c, hsa-let-7e-5p, hsa-let-7f-5p, hsa-let-7g-5p, hsa-let-7i-5p, hsa-miR-106a-5p, hsa-miR-107, hsa-miR-125a-5p, hsa-miR-1275, hsa-miR-130a-3p, hsa-miR-130b-3p, hsa-miR-15a-5p, hsa-miR-15b-5p, hsa-miR-16-5p, hsa-miR-17-5p, hsa-miR-185-5p, hsa-miR-188-5p, hsa-miR-193a-5p, hsa-miR-206, hsa-miR-20a-5p, hsa-miR-296-5p, hsa-miR-30d-5p, hsa-miR-320a, hsa-miR-320b, hsa-miR-320c, hsa-miR-324-3p, hsa-miR-326, hsa-miR-330-3p, hsa-miR-331-3p, hsa-miR-339-5p, hsa-miR-369-3p, hsa-miR-423-5p, hsa-miR-424-5p, hsa-miR-484, hsa-miR-498, hsa-miR-532-3p, hsa-miR-532-5p, hsa-miR-543, hsa-miR-650, hsa-miR-671-5p, hsa-miR-93-5p, and hsa-miR-940.

The 46 miRNAs present in both the InflaRNOme and the AdipoRNOme, and predicted to interact with the anti-inflammatory molecules are: hsa-let-7a-5p, hsa-let-7b-5p, hsa-let-7c, hsa-let-7e-5p, hsa-let-7f-5p, hsa-let-7i-5p, hsa-miR-1, hsa-miR-106a-5p, hsa-miR-107, hsa-miR-125a-5p, hsa-miR-1275, hsa-miR-130a-3p, hsa-miR-130b-3p, hsa-miR-140-3p, hsa-miR-146b-5p, hsa-miR-15a-5p, hsa-miR-15b-5p, hsa-miR-16-5p, hsa-miR-17-5p, hsa-miR-186-5p, hsa-miR-188-5p, hsa-miR-206, hsa-miR-20a-5p, hsa-miR-27a-3p, hsa-miR-27b-3p, hsa-miR-28-3p, hsa-miR-30b-5p, hsa-miR-30d-5p, hsa-miR-320a, hsa-miR-320b, hsa-miR-320c, hsa-miR-326, hsa-miR-330-3p, hsa-miR-342-3p, hsa-miR-361-5p, hsa-miR-362-5p, hsa-miR-423-5p, hsa-miR-424-5p, hsa-miR-498, hsa-miR-532-5p, hsa-miR-543, hsa-miR-618, hsa-miR-650, hsa-miR-671-5p, hsa-miR-93-5p, hsa-miR-940.

Finally, the 34 miRNAs present in both the InflaRNOme and the AdipoRNOme, and predicted predicted to interact with both the pro-inflammatory and the anti-inflammatory molecules are: hsa-let-7a-5p, hsa-let-7b-5p, hsa-let-7c, hsa-let-7e-5p, hsa-let-7f-5p, hsa-let-7i-5p, hsa-miR-106a-5p, hsa-miR-107, hsa-miR-125a-5p, hsa-miR-1275, hsa-miR-130a-3p, hsa-miR-130b-3p, hsa-miR-15a-5p, hsa-miR-15b-5p, hsa-miR-16-5p, hsa-miR-17-5p, hsa-miR-188-5p, hsa-miR-206, hsa-miR-20a-5p, hsa-miR-30d-5p, hsa-miR-320a, hsa-miR-320b, hsa-miR-320c, hsa-miR-326, hsa-miR-330-3p, hsa-miR-423-5p, hsa-miR-424-5p, hsa-miR-498, hsa-miR-532-5p, hsa-miR-543, hsa-miR-650, hsa-miR-671-5p, hsa-miR-93-5p, and hsa-miR-940.

### Example 4

### High-Content Cellular Phenotypic Screening

High-content screening methods are used to screen for novel miRNA agents that modulate the activity of inflammation regulators. High-content screening is a drug discovery method that uses images of living cells to facilitate molecule discovery. Such automated image based screening methods are particularly suitable for identifying agents, which alter cellular phenotypes.

White adipose tissue (WAT) cells contain a single large lipid droplet, whereas, in contrast, brown adipose tissue (BAT) cells contain numerous smaller droplets and a much higher number of mitochondria, which contain iron and make them appear brown. The large number of mitochondria in BAT leads to an increased oxygen consumption, when compared to WAT. Accordingly, it is possible to distinguish between BAT and WAT cells visually based on their cellular phenotype (lipid content and appearance, mitochondria density, oxygen consumption).

Accordingly, high-content screening methods are used to screen for novel miRNA agents that modulate the activity of inflammation regulators. Specifically, the phenotypic appearance of cultured human adipocytes and adipose tissue derived mesenchymal stem cells grown in the presence and absence of miRNA agonists or antagonists is assessed over time by visual inspection of the cultured cells and/or measuring the cellular lipid content (e.g., using Oil Red O Staining or automated high resolution imaging system like CyteSeer 2.0); mitochondrial content (e.g., using Life Technologies Mito-Tracker Red FM), and/or oxygen consumption in vitro (e.g., using the Seahorse Bioscience Extra-Cellular Flux Instrument).

Macrophages display different phenotypes that can switch in response to their microenvironment. In vitro generation of M1 and M2 macrophages is achieved by activating primary monocytes with 100 ng/ml interferon gamma (M1 subtype) or 100 ng/ml IL4 and 50 ng/ml MCSF (M2 subtype). Thus, the phenotypic appearance of cultured macrophages grown in the presence and absence of miRNA agonists or antagonists is assessed over time by looking at their cell surface markers by fluorescence activated cell sorting (FACS). Release of cytokines, miRNA and mRNA profiling are assessed by standard microarray assays.

Co-culture systems are used to study paracrine communications and cross-talk between cell types. For instance, co-culture of differentiated 3T3-L1 adipocytes and macrophage cell line RAW264 results in the upregulation of pro-inflammatory cytokines such as TNF-alpha and the downregulation of the anti-inflammatory cytokine adiponectin. Several co-culture systems are used: 1) human adipocytes and adipose tissue macrophages; 2) human adipocytes and microvascular endothelial cells; and 3) human adipocytes and hepatocytes.

Cells are co-cultured for 24 to 48 hours. FACS is used to characterize the cells before and after co-culture. Lipid droplets are stained with Nile Red. Release of cytokines, miRNA and mRNA profiling are assessed by standard assays.

### Example 5

### Development of Customized Exosomes

This example demonstrates how exosome vesicles that can be customized to specifically deliver their load of miRNA modulators to targeted adipocytes and macrophages while enhancing their intra-cellular penetration, protecting them from degradation, evading rapid clearance by the mononuclear phagocyte system, enhancing passage through fenestrations in the vessel wall and avoiding immune responses.

For the purpose of creating de novo exosomes that can safely and efficiently carry and deliver their miRNA analogs load to adipocytes and related cells, the following criteria are employed (using ExoCarta 2012, a repository database of exosomal proteins, mRNAs, miRNAs and lipids (available online at exocarta.org), Vesiclepedia, a manually curated compendium of molecular data of extracellular vesicles (available online at microvesicles.org) and EVpedia, an integrated and comprehensive proteome, transcriptome, and lipidome database of extracellular vesicles (available online at evpedia.info), as references): (1) Elimination of antigen-presenting molecules to avoid the risk of triggering an innate immune reaction; (2) Restriction of the exosome size to the 30 to 100 nm range, also to avoid the risk of triggering an innate immune reaction and to reduce clearance by the mononuclear phagocyte system; (3) Addition of lipids, lipid rafts and cytoskeleton proteins; (4) Addition of transmembrane proteins like tetraspanins, LAMPs, CD13 and PGRL; (5) Addition of membrane transport and fusion proteins like caveolins; (6) Addition of adipocyte-specific glycosylphosphatidylinositol (GPI)-anchored proteins like CD73; (7) Addition of molecules that are preferentially expressed at the surface of human adipocytes (see Table 2) or at the surface of human macrophages (see Tables 3 and 4).

TABLE 2

| | Name | Entrez Gene ID | Ensembl Gene ID |
|---|---|---|---|
| 1 | Alix (PDCD6IP) | 10015 | ENSG00000170248 |
| 2 | ANXA1 (Annexin 1) | 301 | ENSG00000135046 |
| 3 | ANXA2 (Annexin 2) | 302 | ENSG00000182718 |
| 4 | ANXA4 (Annexin 4) | 307 | ENSG00000196975 |
| 5 | ANXA5 (Annexin 5) | 308 | ENSG00000164111 |
| 6 | ANXA6 (Annexin 6) | 309 | ENSG00000197043 |
| 7 | ANXA7 (Annexin 7) | 310 | ENSG00000138279 |
| 8 | CAV1 (Caveolin 1) | 857 | ENSG00000105974 |
| 9 | CAV2 (Caveolin 2) | 858 | ENSG00000105971 |
| 10 | CD10 (MME) | 4311 | ENSG00000196549 |
| 11 | CD13 (ANPEP) | 290 | ENSG00000166825 |
| 12 | CD146 (MCAM) | 4162 | ENSG00000076706 |
| 13 | CD151 (TSPAN24) | 977 | ENSG00000177697 |
| 14 | CD166 (ALCAM) | 214 | ENSG00000170017 |
| 15 | CD29 (ITGB1) | 3688 | ENSG00000150093 |
| 16 | CD36 (FAT) | 948 | ENSG00000135218 |
| 17 | CD44 (Hyaluronate) | 960 | ENSG00000026508 |
| 18 | CD49e (ITGA4) | 3678 | ENSG00000161638 |
| 19 | CD59 | 966 | ENSG00000085063 |
| 20 | CD63 | 967 | ENSG00000135404 |
| 21 | CD81 (TSPAN28) | 975 | ENSG00000110651 |
| 22 | CD90 (THY1) | 7070 | ENSG00000154096 |
| 23 | CD91 (LRP1) | 4035 | ENSG00000123384 |
| 24 | DCN (Decorin) | 1634 | ENSG00000011465 |
| 25 | DPT (Dermatopontin) | 1805 | ENSG00000143196 |
| 26 | FABP4 | 2167 | ENSG00000170323 |
| 27 | GYPC (Glycophorin C) | 2995 | ENSG00000136732 |
| 28 | ITGA7 | 3679 | ENSG00000135424 |
| 29 | Lamp1 | 3916 | ENSG00000185896 |
| 30 | Mfge8 (Lactadherin) | 4240 | ENSG00000140545 |
| 31 | NPR1 | 4881 | ENSG00000169418 |
| 32 | SLC27A3 (FATP3) | 11000 | ENSG00000143554 |
| 33 | Stomatin | 2040 | ENSG00000148175 |
| 34 | TSPAN3 | 10099 | ENSG00000140391 |
| 35 | TSPAN4 | 7106 | ENSG00000214063 |
| 36 | SXT10 (Syntaxin 10) | 8677 | ENSG00000104915 |

TABLE 3
M1 Macrophage Positive Surface Markers

| | | Entrez | |
|---|---|---|---|
| | Name | Gene ID | Ensembl Gene ID |
| 1 | CCR5 (CD 195) | 1234 | ENSG00000160791 |
| 2 | CCR7 (CD197) | 1236 | ENSG00000126353 |
| 3 | CD14 | 929 | ENSG00000170458 |
| 4 | CD33 | 945 | ENSG00000105383 |
| 5 | CD40 (TNFRSF5) | 958 | ENSG00000101017 |
| 6 | CD68 | 968 | ENSG00000129226 |
| 7 | CD80 | 941 | ENSG00000121594 |
| 8 | CD86 | 942 | ENSG00000114013 |
| 9 | CSF1R (CD115) | 1436 | ENSG00000182578 |
| 10 | EMR1 | 2015 | ENSG00000174837 |
| 11 | Eng (CD105, Endoglin) | 2022 | ENSG00000106991 |
| 12 | FCGR1A (CD64) | 2209 | ENSG00000150337 |
| 13 | FCGR2A (CD32) | 2212 | ENSG00000143226 |
| 14 | FCGR2B (CD32) | 2213 | ENSG00000072694 |
| 15 | FCGR3A (CD16a) | 2214 | ENSG00000203747 |
| 16 | FCGR3B (CD16b) | 2215 | ENSG00000162747 |
| 17 | FUT4 (CD 15) | 2526 | ENSG00000196371 |
| 18 | IL15RA (CD215) | 3601 | ENSG00000134470 |
| 19 | IL1R1 (CD121a) | 3554 | ENSG00000115594 |
| 20 | ITGAL (CD11a, Integrin alpha L) | 3683 | ENSG00000005844 |
| 21 | ITGAM (CD11b, Integrin alpha | 3684 | ENSG00000169896 |
| | M, Mac-1) | | |
| 22 | ITGAX (CD11c, Integrin alpha X) | 3687 | ENSG00000140678 |
| 23 | Lamp2 (CD107b, Mac3) | 3920 | ENSG00000005893 |
| 24 | LGALS3 (MAC2) | 3958 | ENSG00000131981 |
| 25 | PTPRC (CD45) | 5788 | ENSG00000081237 |
| 26 | SLAMF7 (CD319) | 57823 | ENSG00000026751 |
| 27 | TLR2 (CD282) | 7097 | ENSG00000137462 |
| 28 | TLR4 (CD284) | 7099 | ENSG00000136869 |
| 29 | TNFRSF1B (CD120b) | 7133 | ENSG00000028137 |

TABLE 4

| | M2 Macrophage Positive Surface Markers | | |
|---|---|---|---|
| | Name | Entrez Gene ID | Ensembl Gene ID |
| 1 | CD163 | 9332 | ENSG00000177575 |
| 2 | CD1A | 909 | ENSG00000158477 |
| 3 | CD1B | 910 | ENSG00000158485 |
| 4 | CD209 | 30835 | ENSG00000090659 |
| 5 | CD226 | 10666 | ENSG00000150637 |
| 6 | CD302 (DCL-1) | 9936 | ENSG00000241399 |
| 7 | CD68 | 968 | ENSG00000129226 |
| 8 | CD93 | 22918 | ENSG00000125810 |
| 9 | CLEC4A (Dectin-1) | 50856 | ENSG00000111729 |
| 10 | FCER2 (CD23) | 2208 | ENSG00000104921 |
| 11 | IL1R2 (CD121b) | 7850 | ENSG00000115590 |
| 12 | IL27RA | 9466 | ENSG00000104998 |
| 13 | MRC1 (CD206) | 4360 | ENSG00000120586 |

### Example 6

### Proteomic Profiling

Proteomic Profiling is also used to identify novel miRNA targets involved in inflammation. Shotgun proteomics is a method of identifying proteins in complex mixtures using high performance liquid chromatography (HPLC) combined with mass spectrometry (MS). Transfected and transduced cells with miRNA agents and promoter/3′UTR library (as described in Example 4) are harvested and lysed to produce crude soluble (cytosolic) and insoluble (nuclear) fractions. Peptides are from these fractions are then separated by HPLC and analyzed using nanoelectrospray-ionization tandem MS using the isotopic labeling technique SILAC to quantify protein abundance. Spectra are searched against the Ensembl release 54 human protein-coding sequence database using Sequest (Bioworks version 3.3.1, Thermo Scientific).

To avoid missing low abundance proteins, a targeted proteomics approach is also employed to accurately quantify a set of proteins that are known regulators of inflammation (See Table 8). These proteins are analyzed via ELISA based or Luminex based immunoassays using commercially available antibodies.

Optionally, the protein fractions are analyzed using Multiple Reaction Monitoring-Mass Spectrometry on a proteomics platform, whereby only one protein (e.g. IL-6) of the inflammation pathway is accurately quantified using LC-MS-MS.

### Example 7

### In Vitro Validation of the Inflammation Aptamirs

Once the aptamer and miRNA elements are each validated in vitro, their combinations (the aptamirs and exomirs) are validated in vitro, using the same techniques as described above.

### Example 8

### In Vivo Validation of the Thermogenic Aptamirs in Animal Models of Obesity and Visceral Inflammation

Several animal models of obesity have been developed and validated (Kanasaki, et al., 2011; Speakman, et al., 2007). The most commonly used are the Leptin Signaling Defects Lep(ob/ob) and Lepr(db/db) Mouse Models as well as the High-Fat Diet model in C57BL/6J mice (Wang, et al., 2012). This diet-induced obesity (DIO) model closely mimics the increased availability of the high-fat/high-density foods in modern society.

C57B1/6 mice fed a high fat diet are used to assess the efficacy and safety of the thermogenic aptamirs, following the protocol described by Esau, et al., 2006, exploring the effects of miR-122 inhibition on lipid metabolism. Thus, a DIO mouse model is used for in vivo validation of the effectiveness of the miRNA analogs described herein for the increase in thermogenesis and/or the treatment of obesity and other metabolic disorders (Yin, et al., 2013). DIO mice are administered one or more of an agomir, antagomir, aptamir or exomir. Rosiglitazone is used as a positive control. Food intake, blood metabolic parameters, body composition (body weight, body fat, bone mineral and lean mass, body fat distribution, body temperature, O2 consumption and CO2 production, exercise induced thermogenesis, cold induced thermogenesis and resting thermogenesis are measured in the mice prior to and after treatment. A reduction in body mass or body fat or an increase in body temperature or any kind of thermogenesis indicate the in vivo effectiveness of the administered composition.

### REFERENCES

The following references are herein incorporated by reference in their entireties:
Adams, J Am Chem Soc. 105:661, 1983
Alberti, Diabet Med. 15(7):539-553, 1998.
Al-Muhammed, J Microencapsul. 13:293-306, 1996.
Animal Cell Culture R. I. Freshney, ed. 1986.
Armougom, PLoS One. 4(9):e7125, 2009.
Ausubel et al., Current Protocols in Molecular Biology, John Wiley & Sons, Inc., 1994.
Bakhai, Qjm. 101(10):767-776, 2008.
Balkau & Charles. Diabet Med. 16(5):442-443, 1999.
Beaucage, Tetra Lett. 22:1859, 1981
Belousov, Nucleic Acids Res. 25:3440-3444, 1997
Bhatia, Eur Heart J. 33(10):1190-200, 2012.
Blommers, Biochemistry 33:7886-7896, 1994
Bordbar & Palsson. J Intern Med. 271(2):131-141, 2012.
Bordbar, et al., BMC Syst Biol. 5:180, 2011.
Bordbar, et al., Mol Syst Biol. 6:422, 2010.
Bordbar, et al., Mol Syst Biol. 8:558, 2012.
Braasch & Corey, Biochemistry. 41(14):4503-4510, 2002
Brophy, Eur J Clin Pharmacol. 24:103-108, 1983.
Brown, Meth Enzymol. 68:109, 1979
Busch & Zernecke, J Mol Med (Berl). 90(8):877-85, 2012.
Cancello & Clement Bjog. 113(10):1141-1147, 2006.
Cani & Delzenne, Pharmacol Ther. 130(2):202-212, 2011.
Cann, “RNA Viruses: A Practical Approach” Oxford University Press, 2000
Charo & Ransohoff, The New England journal of medicine. 354(6):610-621, 2006.
Chazenbalk, PLoS One. 6(3):e17834, 2011.
Chiang, Int Immunol. 20(2):215-222, 2008.
Cho, et al., Annu Rev Anal Chem. 2:241-264, 2009.
Chonn, Curr Opin Biotechnol. 6:698-708, 1995;
Chung, Ageing Res Rev. 8(1):18-30, 2009.
Chung, et al., J Dent Res. 90(7):830-840, 2011.
Coffin et al., Retroviruses. 1997
Collino, et al., PLoS One. 5(7):e11803, 2010.
Creemers, et al., Circ Res. 110(3):483-495, 2012.
Crooke et al, J. Pharmacol. Exp. Ther., 1996, 277, 923-937
Dalmas, et al., Trends Immunol. 32(7):307-314, 2011.
De Mesmaeker et al. Ace Chem Res. 28:366-374, 1995
Diamant, et al., Obesity reviews. 12(4):272-281, 2011.
Donath & Shoelson, Nat Rev Immunol. 11(2):98-107, 2011.
Duarte, et al. PNAS USA. 104(6):1777-1782, 2007.
Ebert et al., Nature Methods. 4(9):721-726, 2007
Elabd, et al., Biochemical and biophysical research communications. 361(2):342-348, 2007.
Elabd, et al., Stem Cells. 27(11):2753-2760, 2009.
Elmen, et al, Nature. 452:896-899, 2008.
Esau, et al, Cell Metabolism. 3(2):87-98, 2006.
Estep, et al., Aliment Pharmacol Ther. 32(3):487-497, 2010.
Eyles, J. Pharm. Pharmacol. 49:669-674, 1997.
Fang & Tan, Acc Chem Res. 43(1):48-57, 2010.
Fotherby, Contraception. 54:59-69, 1996.
Frazier, et al., JPEN J Parenter Enteral Nutr. 35(5 Suppl):14S-20S, 2011.
Frenkel, Free Radic Biol Med. 19:373-380, 1995
Friedman, et al., Bioinformatics. 26(15):1920-1921, 2010.
Gait, Oligonucleotide Synthesis, 1984.
Gallagher, et al., Genome medicine. 2(2):9, 2010.
Gao, Pharm Res. 12:857-863, 1995.
Gebeyehu, et al. Nucl Acids Res. 15:4513, 1987.
Genesis. 30(3), 2001.
Glover, DNA Cloning: A Practical Approach, Volumes I and II, 1985.
Groning, Pharmazie. 51:337-341, 1996.
Gui, et al., Mediators Inflamm. 2012:693083, 2012.
Hall, et al., Curr Protoc Nucleic Acid Chem. 9(3):1-27, 2010.
Hames & Higgins, Nucleic Acid Hybridization, 1985.
Hames & Higgins, Transcription And Translation 1984.
Heasman, J Dev Biol. 243:209-214, 2002.
Henrichot, et al., Arterioscler Thromb Vasc Biol. 25(12):2594-2599, 2005.
Hidalgo-Aragones, J Steroid Biochem Mol Biol. 58:611-617, 1996.
Hirsch, Trends Plant Sci. 17(3):123-125, 2012.
Hotamisligil, et al., Nature. 444(7121):860-867, 2006.
Hsu, et al., Nucleic acids research. 39(Database issue):D163-169, 2011.
Hulsmans, et al., FASEB J. 25:2515-2527, 2011.
Hunter, et al., PLoS One. 3(11):e3694, 2008.
Illouz, et al., Adipose Stem Cells and Regenerative Medicine. 133-139, 2011.
Immobilized Cells And Enzymes IRL Press, 1986.
Inadera, Int J Med Sci. 5(5):248-262, 2008.
International Application Serial No. PCT/US2013/037579
International Application Serial No. PCT/US2013/053613
Ito, et al., Int J Inflam. 2011:720926, 2011.
Johnson, J Pharm Sci. 84:1144-1146, 1995
Kabanov et al, FEBS Lett. 1259:327-330, 1990.
Kanwar, et al., Crit Rev Biochem Mol Biol. 46(6):459-477, 2011.
Kashtan, et al., Phys Rev E Stat Nonlin Soft Matter Phys. 70(3 Pt 1):031909, 2004.
Keefe, et al., Nat Rev Drug Discov. 9(7):537-550, 2010.
Kootte, et al., Diabetes Obes Metab. 14(2):112-120, 2012.
Kornberg, DNA Replication, W. H. Freeman & Co.: San Francisco. pp75-77, 1980
Krutzfeldt, et al, Nature. 438:685-689, 2005.
Lacerra et al, Proc Natl Acad Sci. 97:9591-9596, 2000.
Lassalle, et al., Curr Drug Metab. 13(8):1130-44, 2012.
Letsinger et al, Proc. Natl. Acad. Sci. USA. 86:6553-6556, 1989.
Ley, Curr Opin Gastroenterol. 26(1):5-11, 2010.
Lin, et al., BMC systems biology. 6:18, 2012.
Lindroos, et al., Stem Cell Rev. 7(2):269-291, 2011.
Liu & Rennard, Mol Cell Pharmacol. 3(3):143-151, 2011.
Lumeng & Saltiel, J Clin Invest. 121(6):2111-2117, 2011.
Mancharan et al, Nucleosides & Nucleotides, 14:969-97, 1995.
Mandard, The Journal of biological chemistry. 281(2):934-944, 2006.
Mangan & Alon, PNAS USA. 100(21):11980-11985, 2003.
Manoharan et al, Ann. N.Y. Acad. Sci. 660:306-309, 1992.
Manoharan et al, Bioorg. Med. Chem. Let. 3:2765-2770, 1993.
Manoharan et al, Bioorg. Med. Chem. Let. 4:1053-1060, 1994.
Manoharan et al, Tetrahedron Lett. 36:3651-3654, 1995.
Manoharan et al., Tetrahedron Lett. 36:3651-3654, 1995.
Martin et al, Helv. Chim. Acta. 78:486, 1995.
Mause & Weber, Circ Res. 107(9):1047-1057, 2010.
Minto, J. Pharmacol. Exp. Ther. 281:93-102, 1997.
Mishra et al, Biochim. Biophys. Acta. 1264:229-237, 1995.
Mottillo, et al., J Am Coll Cardiol. 56(14):1113-1132, 2010.
Muller, et al., Cell Signal. 23(7):1207-1223, 2011.
Murdolo & Smith, Nutr Metab Cardiovasc Dis. 16 Supp11:S35-38, 2006.
Musso, et al., Current opinion in lipidology. 21(1):76-83, 2010.
Narang, Meth Enzymol. 68:90, 1979
Nasevicius, et al, Nat Genet. 26:216-220, 2000.
Nazari-Jahantigh, et al., Thromb Haemost. 107(4):611-618, 2012.
Neels, et al., J Clin Invest. 116(1):33-35, 2006.
Neff, et al., Sci Transl Med. 3(66):66ra66, 2011.
Nielsen et al, Science. 254:1497-1500, 1991.
Oberhauser et al., Nucl. Acids Res. 20:533-538, 1992.
Orbay, et al., Stem Cells Int. 2012:461718, 2012.
Ostro, Am. J. Hosp. Pharm. 46:1576-1587, 1989.
Ouchi, et al., Nat Rev Immunol. 11(2):85-97, 2011.
Perbal, A Practical Guide To Molecular Cloning, 1984.
Petrovic, et al., J Biol Chem. 285(10):7153-7164, 2010.
Pisani, et al., Front Endocrinol (Lausanne) 2:87, 2011.
Povsic, et al., Journal of cardiovascular translational research. 3(6):704-716, 2010.
Rao, J. Biomater Sci. Polym. Ed. 7:623-645, 1995.
Ray, et al., Pharmaceuticals. 3:1761-1778, 2010.
Rayner, et al., Thromb Haemost. 107(4):642-647, 2012.
Remington: The Science and Practice of Pharmacy, 21st ed., 2005
Rieger, et al., Frontiers in Genetics. 2:1-6, 2011.
Rohatagi, J. Clin. Pharmacol. 35:1187-1193, 1995.
Rohatagi, Pharmazie. 50:610-613, 1995.
Sambrook, Fritsch & Maniatis, Molecular Cloning: A Laboratory Manual, Second Edition (1989) Cold Spring Harbor Laboratory Press, Cold Spring Harbor:N.Y., 1989.
Sanghvi, et al., Antisense Research and Applications, CRC Press:Boca Raton, 276-278, 2012
Schober, et al., Thromb Haemost. 107(4):603-604, 2012.
Sefah, et al., Nat Protoc. 5(6):1169-1185, 2010.
Shea et al, Nucl. Acids Res. 18:3777-3783, 1990.
Suganami, et al., Arterioscler Thromb Vasc Biol. 25(10):2062-2068, 2005.
Suganami, et al., J Leukoc Biol. 88(1):33-39, 2010.
Sun, et al., J Clin Invest. 121(6):2094-2101, 2011.
Svinarchuk et al, Biochimie. 75:49-54, 1993.
Tjwa, Ann. Allergy Asthma Immunol. 75:107-111, 1995.
Turnbaugh, et al., Nature. 457(7228):480-484, 2009.
U.S. 20040028670
U.S. Pat. No. 3,687,808
U.S. Pat. No. 4,458,066
U.S. Pat. No. 4,469,863
U.S. Pat. No. 4,476,301
U.S. Pat. No. 4,587,044
U.S. Pat. No. 4,605,735
U.S. Pat. No. 4,667,025
U.S. Pat. No. 4,762,779
U.S. Pat. No. 4,789,737
U.S. Pat. No. 4,824,941
U.S. Pat. No. 4,828,979
U.S. Pat. No. 4,835,263
U.S. Pat. No. 4,845,205
U.S. Pat. No. 4,876,335
U.S. Pat. No. 4,904,582
U.S. Pat. No. 4,948,882
U.S. Pat. No. 4,958,013
U.S. Pat. No. 5,013,830
U.S. Pat. No. 5,023,243
U.S. Pat. No. 5,034,506
U.S. Pat. No. 5,034,506
U.S. Pat. No. 5,082,830
U.S. Pat. No. 5,109,124
U.S. Pat. No. 5,112,963
U.S. Pat. No. 5,118,802
U.S. Pat. No. 5,130,302
U.S. Pat. No. 5,134,066
U.S. Pat. No. 5,138,045
U.S. Pat. No. 5,149,797
U.S. Pat. No. 5,166,315
U.S. Pat. No. 5,175,273
U.S. Pat. No. 5,177,196
U.S. Pat. No. 5,185,444
U.S. Pat. No. 5,188,897
U.S. Pat. No. 5,214,134
U.S. Pat. No. 5,214,136
U.S. Pat. No. 5,216,141
U.S. Pat. No. 5,218,105
U.S. Pat. No. 5,220,007
U.S. Pat. No. 5,235,033
U.S. Pat. No. 5,245,022
U.S. Pat. No. 5,254,469
U.S. Pat. No. 5,256,775
U.S. Pat. No. 5,258,506
U.S. Pat. No. 5,262,536
U.S. Pat. No. 5,264,423
U.S. Pat. No. 5,264,562
U.S. Pat. No. 5,264,564
U.S. Pat. No. 5,272,250
U.S. Pat. No. 5,276,019
U.S. Pat. No. 5,278,302
U.S. Pat. No. 5,286,717
U.S. Pat. No. 5,292,873
U.S. Pat. No. 5,317,098
U.S. Pat. No. 5,321,131
U.S. Pat. No. 5,366,878
U.S. Pat. No. 5,367,066
U.S. Pat. No. 5,371,241
U.S. Pat. No. 5,391,723
U.S. Pat. No. 5,399,676
U.S. Pat. No. 5,403,711
U.S. Pat. No. 5,405,938
U.S. Pat. No. 5,405,939
U.S. Pat. No. 5,414,077
U.S. Pat. No. 5,416,203
U.S. Pat. No. 5,432,272
U.S. Pat. No. 5,434,257
U.S. Pat. No. 5,451,463
U.S. Pat. No. 5,453,496
U.S. Pat. No. 5,455,233
U.S. Pat. No. 5,457,187
U.S. Pat. No. 5,459,255
U.S. Pat. No. 5,466,677
U.S. Pat. No. 5,470,967
U.S. Pat. No. 5,476,925
U.S. Pat. No. 5,484,908
U.S. Pat. No. 5,486,603
U.S. Pat. No. 5,489,677
U.S. Pat. No. 5,491,133
U.S. Pat. No. 5,502,177
U.S. Pat. No. 5,510,475
U.S. Pat. No. 5,512,439
U.S. Pat. No. 5,512,667
U.S. Pat. No. 5,514,785
U.S. Pat. No. 5,519,126
U.S. Pat. No. 5,525,465
U.S. Pat. No. 5,525,711
U.S. Pat. No. 5,536,821
U.S. Pat. No. 5,539,082
U.S. Pat. No. 5,541,306
U.S. Pat. No. 5,541,307
U.S. Pat. No. 5,541,313
U.S. Pat. No. 5,545,730
U.S. Pat. No. 5,550,111
U.S. Pat. No. 5,552,538
U.S. Pat. No. 5,552,540
U.S. Pat. No. 5,561,225
U.S. Pat. No. 5,563,253
U.S. Pat. No. 5,565,350
U.S. Pat. No. 5,565,552
U.S. Pat. No. 5,567,810
U.S. Pat. No. 5,571,799
U.S. Pat. No. 5,574,142
U.S. Pat. No. 5,578,717
U.S. Pat. No. 5,578,718
U.S. Pat. No. 5,580,731
U.S. Pat. No. 5,585,481
U.S. Pat. No. 5,587,361
U.S. Pat. No. 5,587,371
U.S. Pat. No. 5,587,469
U.S. Pat. No. 5,591,584
U.S. Pat. No. 5,595,726
U.S. Pat. No. 5,596,086
U.S. Pat. No. 5,596,091
U.S. Pat. No. 5,597,696
U.S. Pat. No. 5,599,923
U.S. Pat. No. 5,599,928
U.S. Pat. No. 5,602,240
U.S. Pat. No. 5,608,046
U.S. Pat. No. 5,610,289
U.S. Pat. No. 5,614,617
U.S. Pat. No. 5,618,704
U.S. Pat. No. 5,623,065
U.S. Pat. No. 5,623,070
U.S. Pat. No. 5,625,050
U.S. Pat. No. 5,633,360
U.S. Pat. No. 5,652,355
U.S. Pat. No. 5,652,356
U.S. Pat. No. 5,663,312
U.S. Pat. No. 5,677,437
U.S. Pat. No. 5,677,439
U.S. Pat. No. 5,681,941
U.S. Pat. No. 5,688,941
U.S. Pat. No. 5,700,922
U.S. Pat. No. 5,714,331
U.S. Pat. No. 5,716,928
U.S. Pat. No. 5,719,262
U.S. Pat. No. 5,750,692
U.S. Pat. No. 5,858,401
U.S. Pat. No. 6,007,839
U.S. Pat. No. 6,063,400
U.S. Pat. No. 6,107,094
van den Boom, et al., Nature biotechnology. 29(4):325-326, 2011.
Vickers & Remaley, Curr Opin Lipidol. 23(2):91-97, 2012.
Wang et al, J Am Chem Soc. 122:8595-8602, 2000.
Wang, et al., Curr Med Chem. 18(27):4169-4174, 2011.
Woodard, et al., J Gastrointest Surg. 13(7):1198-1204, 2009.
Wu, et al., Cell. 150(2):366-76, 2012.
Xiao, et al., Journal of Cellular Physiology. 212(2):285-292, 2007
Zampetaki, et al., Cardiovascular research. 93(4):555-562, 2012.
Zemecke, et al., Thromb Haemost. 107(4):626-633, 2012.
Zhang, et al., Cell Res. 22(1):107-126, 2012.
Zhou & Rossi, Curr Top Med Chem. 9(12):1144-1157, 2009.
Zhou & Rossi, Oligonucleotides. 21(1):1-10, 2011.
Zhou & Rossi, Silence. 1(1):4, 2010.
Zhuang, et al., Adv Drug Deliv Rev. 65(3):342-7, 2012.
Zhuang, et al., Circulation. 125(23):2892-2903, 2012.

1. A method of modulating the inflammatory activity of a cell associated with chronic visceral inflammation or modulating chronic visceral inflammation in a tissue, the method comprising contacting the cell or tissue with a miRNA agent that modulates the activity of at least one inflammation regulator in the cell.

2. The method of claim 1, wherein the cell associated with chronic visceral inflammation is a pre-adipocyte, adipocyte, adipose tissue derived mesenchymal stem cell, stroma vascular cell, macrophage, lymphocyte, endothelial cell, vascular cell, fibroblast, hepatocyte, myocyte, or a precursor thereof.

3-5. (canceled)

6. The method of claim 3, wherein the inflammation regulator is selected from the group consisting of AICDA, AIM2, AKT2, ANGPTL2 (angiopoietin-like 2), CASP1 (Caspase 1), CCL2 (Chemokine C-C motif ligand 2, MCP-1), CCL3 (MIP-1 alpha), CCL4 (MIP-1 beta), CCL5 (RANTES), CCL7 (MCP-3), CCL8 (MCP-2), CCL11 (Eotaxin), CCL15 (MIP-1 delta), CCL17 (TARC), CCL19 (MIP-3b), CCL20 (MIP-3-alpha), CCL22 (MDC), CCR2 (Chemokine C-C motif receptor 2, MCP-1-R), CCR7 (CD197), CD40LG (CD154), CD69, CEBPA, CEBPB, CFD (Adipsin), CHUK (IKKA), CXCR4 (Chemokine C-X-C motif receptor 4, CD184), MT-CO2 (Cytochrome c oxidase subunit II), CRP, CXCL1, CXCL2, CXCL3, CXCL5, CXCL9 (MIG), CXCL10 (IP-10), CXCL11 (ITAC), CXCL12 (SDF-1), CXCL13 (BCA1), CXCL16, FGA (Fibrinogen A), FGB (Fibrinogen B), FGG (Fibrinogen G), HIF1A, ICAM1 (CD54), IFNB1, IFNG, IKBKB (IKKB), IKBKE (IKKE), IL12A, IL12B, IL15, IL17A, IL18, IL1A, IL1B, IL2, IL23A, IL6, IL8 (CXCL8), INPP5D, IRAK1, IRAK2, IRF3 (Interferon-regulatory factor 3), IRF4 (Interferon-regulatory factor 4), IRF5 (Interferon-regulatory factor 5), LCN2 (Lipocalin 2), LEP (Leptin), MAP3K7, MMP2, MMP9, MYD88, NAMPT (Visfatin), NFKB1, NLRP3 (NLR family, pyrin domain containing 3), NOS2 (iNOS), SPP1 (Osteopontin), P2RX7 (Purinergic receptor P2X7), PDCD4, PKNOX1, PTX3 (Pentraxin 3), RBP4, RETN (Resistin), SAA1 (serum amyloid A1), SELE (E-selectin), SELP (P-selectin), SERPINE1 (plasminogen activator inhibitor type 1), SOCS1, SOCS3, SPI1, STAT1, TAB1, TAB2, TGFB1, TIRAP, TLR2 (CD282), TLR3 (CD283), TLR4 (CD284), TLR6 (CD286), TLR7, TLR8 (CD288), TNF (TNF alpha), TRAF6, TXNIP (Thioredoxin interacting protein), VCAM1, VEGFA, WNT5A, ADIPOQ (Adiponectin), AKT1, ALOX15, ARG1 (Arginase 1), ARG2 (Arginase 2), CCL16, CCL18, CCL24, CD36, CTBP1, CXCR1, CXCR2, FABP4, HDAC3, IL10, IL10RA (IL10R), IL13, IL1RN (IL1RA), IL3, IL33, IL4, IL4R, IL5, KDM6B (JMJD3), KLF2, KLF4, MAF (c-MAF), MRC1 (Macrophage mannose receptor 1), MT-COX3, MYC (c-Myc), NR1H3 (LXRA), PPARD, PPARG, PPARGC1A, PRDX3 (Peroxiredoxin), RETNLB (FIZZ1), SCARB1, SFRP5, SIRT1, SIRT2, SIRT3, SIRT4, SIRT5, SIRT6, SIRT7, SOCS2, STAT3, and STAT6.

7-15. (canceled)

16. The method of claim 1, wherein the miRNA agent is a miRNA selected from the group consisting of the miRNA is selected from the group consisting of hsa-let-7a-5p, hsa-let-7b-5p, hsa-let-7c, hsa-let-7d-3p, hsa-let-7d-5p, hsa-let-7e-5p, hsa-let-7f-5p, hsa-let-7g-5p, hsa-let-7i-5p, hsa-miR-1, hsa-miR-10a-5p, hsa-miR-100-5p, hsa-miR-103a-2-5p, hsa-miR-103a-3p, hsa-miR-106a-5p, hsa-miR-106b-5p, hsa-miR-107, hsa-miR-122-5p, hsa-miR-124-3p, hsa-miR-1246, hsa-miR-1249, hsa-miR-125a-5p, hsa-miR-125b-5p, hsa-miR-126-3p, hsa-miR-1268a, hsa-miR-1275, hsa-miR-128-3p, hsa-miR-1281, hsa-miR-1307-3p, hsa-miR-130a-3p, hsa-miR-130b-3p, hsa-miR-132-3p, hsa-miR-132-5p, hsa-miR-133a-5p, hsa-miR-133b, hsa-miR-134-5p, hsa-miR-135a-5p, hsa-miR-138-5p, hsa-miR-139-3p, hsa-miR-140-3p, hsa-miR-143-3p, hsa-miR-145-5p, hsa-miR-1469, hsa-miR-146a-5p, hsa-miR-146b-5p, hsa-miR-147a, hsa-miR-148a-3p, hsa-miR-149-5p, hsa-miR-150-3p, hsa-miR-150-5p, hsa-miR-151a-3p, hsa-miR-151a-5p, hsa-miR-152-3p, hsa-miR-155-5p, hsa-miR-15a-5p, hsa-miR-15b-5p, hsa-miR-16-5p, hsa-miR-17-5p, hsa-miR-181a-3p, hsa-miR-181a-5p, hsa-miR-181b-3p, hsa-miR-181b-5p, hsa-miR-181c-5p, hsa-miR-182-5p, hsa-miR-183-5p, hsa-miR-185-5p, hsa-miR-186-5p, hsa-miR-188-5p, hsa-miR-190b, hsa-miR-191-5p, hsa-miR-1915-3p, hsa-miR-192-5p, hsa-miR-193a-5p, hsa-miR-193b-3p, hsa-miR-197-3p, hsa-miR-19a-3p, hsa-miR-19b-3p, hsa-miR-206, hsa-miR-20a-5p, hsa-miR-21-5p, hsa-miR-210-5p, hsa-miR-212-3p, hsa-miR-214-3p, hsa-miR-22-3p, hsa-miR-221-3p, hsa-miR-222-3p, hsa-miR-223-3p, hsa-miR-23a-3p, hsa-miR-23a-5p, hsa-miR-23b-3p, hsa-miR-24-2-5p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-296-5p, hsa-miR-299-5p, hsa-miR-29a-3p, hsa-miR-29b-3p, hsa-miR-29c-5p, hsa-miR-2c-3p, hsa-miR-30a-5p, hsa-miR-30b-5p, hsa-miR-30c-5p, hsa-miR-30d-5p, hsa-miR-31-5p, hsa-miR-320a, hsa-miR-320b, hsa-miR-320c, hsa-miR-320d, hsa-miR-323a-3p, hsa-miR-324-3p, hsa-miR-324-5p, hsa-miR-326, hsa-miR-328-3p, hsa-miR-330-3p, hsa-miR-331-3p, hsa-miR-337-3p, hsa-miR-337-5p, hsa-miR-339-3p, hsa-miR-339-5p, hsa-miR-33a-5p, hsa-miR-342-3p, hsa-miR-345-5p, hsa-miR-34a-5p, hsa-miR-361-5p, hsa-miR-362-5p, hsa-miR-369-3p, hsa-miR-370-3p, hsa-miR-371a-3p, hsa-miR-374a-5p, hsa-miR-378a-3p, hsa-miR-378a-5p, hsa-miR-421, hsa-miR-423-5p, hsa-miR-424-5p, hsa-miR-425-3p, hsa-miR-425-5p, hsa-miR-432-5p, hsa-miR-433-3p, hsa-miR-483-3p, hsa-miR-483-5p, hsa-miR-484, hsa-miR-485-3p, hsa-miR-487a-3p, hsa-miR-498, hsa-miR-500a-3p, hsa-miR-502-3p, hsa-miR-502-5p, hsa-miR-503-5p, hsa-miR-505-3p, hsa-miR-505-5p, hsa-miR-51 1-3p, hsa-miR-516a-3p, hsa-miR-517-5p, hsa-miR-517c-3p, hsa-miR-518e-3p, hsa-miR-518f-3p, hsa-miR-519b-3p, hsa-miR-519d-3p, hsa-miR-522-3p, hsa-miR-532-3p, hsa-miR-532-5p, hsa-miR-543, hsa-miR-550a-3p, hsa-miR-572, hsa-miR-573, hsa-miR-574-3p, hsa-miR-576-3p, hsa-miR-582-5p, hsa-miR-604, hsa-miR-610, hsa-miR-618, hsa-miR-622, hsa-miR-625-5p, hsa-miR-629-3p, hsa-miR-629-5p, hsa-miR-630, hsa-miR-638, hsa-miR-642a-5p, hsa-miR-643, hsa-miR-650, hsa-miR-652-3p, hsa-miR-656-3p, hsa-miR-663a, hsa-miR-671-3p, hsa-miR-671-5p, hsa-miR-7-5p, hsa-miR-744-5p, hsa-miR-766-3p, hsa-miR-875-5p, hsa-miR-877-5p, hsa-miR-885-5p, hsa-miR-9-5p, hsa-miR-92a-3p, hsa-miR-92b-3p, hsa-miR-93-5p, hsa-miR-940, hsa-miR-941, hsa-miR-942-5p, hsa-miR-96-5p, hsa-miR-99a-5p, and hsa-miR-99b-5p or an analog, agomir, or antagomir thereof.

17. The method of claim 1, wherein the miRNA agent is a miRNA selected from the group consisting of the miRNA is selected from the group consisting of hsa-let-7a-5p, hsa-let-7b-5p, hsa-let-7c, hsa-let-7d-3p, hsa-let-7d-5p, hsa-let-7e-5p, hsa-let-7f-5p, hsa-let-7g-5p, hsa-let-7i-5p, hsa-miR-1, hsa-miR-100-5p, hsa-miR-103a-2-5p, hsa-miR-103a-3p, hsa-miR-106a-5p, hsa-miR-106b-5p, hsa-miR-107, hsa-miR-10a-5p, hsa-miR-122-5p, hsa-miR-124-3p, hsa-miR-1246, hsa-miR-1249, hsa-miR-125a-5p, hsa-miR-125b-5p, hsa-miR-126-3p, hsa-miR-1268a, hsa-miR-1275, hsa-miR-130a-3p, hsa-miR-130b-3p, hsa-miR-132-3p, hsa-miR-132-5p, hsa-miR-133b, hsa-miR-135a-5p, hsa-miR-138-5p, hsa-miR-139-3p, hsa-miR-140-3p, hsa-miR-143-3p, hsa-miR-145-5p, hsa-miR-146a-5p, hsa-miR-146b-5p, hsa-miR-147a, hsa-miR-148a-3p, hsa-miR-149-5p, hsa-miR-150-3p, hsa-miR-150-5p, hsa-miR-151a-3p, hsa-miR-151a-5p, hsa-miR-155-5p, hsa-miR-15a-5p, hsa-miR-15b-5p, hsa-miR-16-5p, hsa-miR-17-5p, hsa-miR-181a-3p, hsa-miR-181a-5p, hsa-miR-181b-5p, hsa-miR-181c-5p, hsa-miR-182-5p, hsa-miR-183-5p, hsa-miR-185-5p, hsa-miR-186-5p, hsa-miR-188-5p, hsa-miR-190b, hsa-miR-191-5p, hsa-miR-1915-3p, hsa-miR-192-5p, hsa-miR-193a-5p, hsa-miR-193b-3p, hsa-miR-197-3p, hsa-miR-19a-3p, hsa-miR-19b-3p, hsa-miR-206, hsa-miR-20a-5p, hsa-miR-21-5p, hsa-miR-212-3p, hsa-miR-214-3p, hsa-miR-22-3p, hsa-miR-221-3p, hsa-miR-222-3p, hsa-miR-223-3p, hsa-miR-23a-3p, hsa-miR-23a-5p, hsa-miR-23b-3p, hsa-miR-24-2-5p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-296-5p, hsa-miR-299-5p, hsa-miR-29a-3p, hsa-miR-29b-3p, hsa-miR-29c-5p, hsa-miR-30a-5p, hsa-miR-30b-5p, hsa-miR-30c-5p, hsa-miR-30d-5p, hsa-miR-31-5p, hsa-miR-320a, hsa-miR-320b, hsa-miR-320c, hsa-miR-323a-3p, hsa-miR-324-3p, hsa-miR-324-5p, hsa-miR-326, hsa-miR-330-3p, hsa-miR-331-3p, hsa-miR-337-3p, hsa-miR-337-5p, hsa-miR-339-3p, hsa-miR-339-5p, hsa-miR-33a-5p, hsa-miR-342-3p, hsa-miR-345-5p, hsa-miR-34a-5p, hsa-miR-361-5p, hsa-miR-362-5p, hsa-miR-369-3p, hsa-miR-37 1a-3p, hsa-miR-374a-5p, hsa-miR-378a-3p, hsa-miR-378a-5p, hsa-miR-421, hsa-miR-423-5p, hsa-miR-424-5p, hsa-miR-425-3p, hsa-miR-425-5p, hsa-miR-432-5p, hsa-miR-483-3p, hsa-miR-483-5p, hsa-miR-484, hsa-miR-498, hsa-miR-500a-3p, hsa-miR-502-3p, hsa-miR-502-5p, hsa-miR-503-5p, hsa-miR-505-3p, hsa-miR-505-5p, hsa-miR-518e-3p, hsa-miR-518f-3p, hsa-miR-532-3p, hsa-miR-532-5p, hsa-miR-543, hsa-miR-550a-3p, hsa-miR-572, hsa-miR-574-3p, hsa-miR-576-3p, hsa-miR-582-5p, hsa-miR-618, hsa-miR-625-5p, hsa-miR-629-3p, hsa-miR-629-5p, hsa-miR-630, hsa-miR-638, hsa-miR-642a-5p, hsa-miR-643, hsa-miR-650, hsa-miR-652-3p, hsa-miR-663a, hsa-miR-671-5p, hsa-miR-7-5p, hsa-miR-744-5p, hsa-miR-877-5p, hsa-miR-9-5p, hsa-miR-92a-3p, hsa-miR-92b-3p, hsa-miR-93-5p, hsa-miR-940, hsa-miR-941, hsa-miR-96-5p, hsa-miR-99a-5p, and hsa-miR-99b-5p or an analog, agomir, or antagomir thereof.

18. The method of claim 1, wherein the miRNA agent is a miRNA selected from the group consisting of the miRNA is selected from the group consisting of hsa-let-7a-5p, hsa-let-7b-5p, hsa-let-7c, hsa-let-7e-5p, hsa-let-7f-5p, hsa-let-7g-5p, hsa-let-7i-5p, hsa-miR-106a-5p, hsa-miR-107, hsa-miR-125a-5p, hsa-miR-1275, hsa-miR-130a-3p, hsa-miR-130b-3p, hsa-miR-15a-5p, hsa-miR-15b-5p, hsa-miR-16-5p, hsa-miR-17-5p, hsa-miR-185-5p, hsa-miR-188-5p, hsa-miR-193a-5p, hsa-miR-206, hsa-miR-20a-5p, hsa-miR-296-5p, hsa-miR-30d-5p, hsa-miR-320a, hsa-miR-320b, hsa-miR-320c, hsa-miR-324-3p, hsa-miR-326, hsa-miR-330-3p, hsa-miR-331-3p, hsa-miR-339-5p, hsa-miR-369-3p, hsa-miR-423-5p, hsa-miR-424-5p, hsa-miR-484, hsa-miR-498, hsa-miR-532-3p, hsa-miR-532-5p, hsa-miR-543, hsa-miR-650, hsa-miR-671-5p, hsa-miR-93-5p, and hsa-miR-940 or an analog, agomir, or antagomir thereof.

19. The method of claim 1, wherein the miRNA agent is a miRNA selected from the group consisting of the miRNA is selected from the group consisting of hsa-let-7a-5p, hsa-let-7b-5p, hsa-let-7c, hsa-let-7e-5p, hsa-let-7f-5p, hsa-let-7i-5p, hsa-miR-1, hsa-miR-106a-5p, hsa-miR-107, hsa-miR-125a-5p, hsa-miR-1275, hsa-miR-130a-3p, hsa-miR-130b-3p, hsa-miR-140-3p, hsa-miR-146b-5p, hsa-miR-15a-5p, hsa-miR-15b-5p, hsa-miR-16-5p, hsa-miR-17-5p, hsa-miR-186-5p, hsa-miR-188-5p, hsa-miR-206, hsa-miR-20a-5p, hsa-miR-27a-3p, hsa-miR-27b-3p, hsa-miR-28-3p, hsa-miR-30b-5p, hsa-miR-30d-5p, hsa-miR-320a, hsa-miR-320b, hsa-miR-320c, hsa-miR-326, hsa-miR-330-3p, hsa-miR-342-3p, hsa-miR-361-5p, hsa-miR-362-5p, hsa-miR-423-5p, hsa-miR-424-5p, hsa-miR-498, hsa-miR-532-5p, hsa-miR-543, hsa-miR-618, hsa-miR-650, hsa-miR-671-5p, hsa-miR-93-5p, hsa-miR-940 or an analog, agomir, or antagomir thereof.

20. The method of claim 1, wherein the miRNA agent is a miRNA selected from the group consisting of the miRNA is selected from the group consisting of hsa-let-7a-5p, hsa-let-7b-5p, hsa-let-7c, hsa-let-7e-5p, hsa-let-7f-5p, hsa-let-7i-5p, hsa-miR-106a-5p, hsa-miR-107, hsa-miR-125a-5p, hsa-miR-1275, hsa-miR-130a-3p, hsa-miR-130b-3p, hsa-miR-15a-5p, hsa-miR-15b-5p, hsa-miR-16-5p, hsa-miR-17-5p, hsa-miR-188-5p, hsa-miR-206, hsa-miR-20a-5p, hsa-miR-30d-5p, hsa-miR-320a, hsa-miR-320b, hsa-miR-320c, hsa-miR-326, hsa-miR-330-3p, hsa-miR-423-5p, hsa-miR-424-5p, hsa-miR-498, hsa-miR-532-5p, hsa-miR-543, hsa-miR-650, hsa-miR-671-5p, hsa-miR-93-5p, and hsa-miR-940 or an analog, agomir, or antagomir thereof.

21. (canceled)

22. The method of claim 1, wherein the miRNA agent is linked to a targeting moiety.

23. The method of claim 22, wherein the targeting moiety is an aptamer.

24. The method of claim 22, wherein the targeting moiety delivers the miRNA agent to a specific cell type or tissue.

25. The method of claim 22, wherein the targeting moiety delivers the miRNA agent to a pre-adipocyte, adipocyte, adipose tissue derived mesenchymal stem cell, stroma vascular cell, macrophage, lymphocyte, endothelial cell, vascular cell, fibroblast, hepatocyte, myocyte, or a precursor thereof.

26. The method of claim 22, wherein the targeting moiety binds to at least one cell surface molecule selected from the group consisting of Alix (PDCD6IP), ANXA1 (Annexin 1), ANXA2 (Annexin 2), ANXA4 (Annexin 4), ANXA5 (Annexin 5), ANXA6 (Annexin 6), ANXA7 (Annexin 7), Caveolin 1 (CAV1), Caveolin 2 (CAV2), CD10 (MME), CD13 (ANPEP), CD146 (MCAM), CD151 (TSPAN24), CD166 (ALCAM), CD29 (ITGB1), CD36 (FAT), CD44 (Hyaluronate), CD49e (ITGA4), CD63, CD81 (TSPAN28), CD90 (THY1), CD91 (LRP1), DPT (Dermatopontin), FABP4, GYPC (Glycophorin C), ITGA7 (integrin, alpha 7), Lamp1, Mfge8 (Lactadherin), NPR1, Stomatin, SXT10 (Syntaxin 10), TSPAN3, TSPAN4, CCR5 (CD195), CCR7 (CD197), CD14, CD33, CD40 (TNFRSF5), CD68, CD80, CD86, CSF1R (CD115), EMR1, Eng (CD105, Endoglin), FCGR1A (CD64), FCGR2A (CD32), FCGR2B (CD32), FCGR3A (CD16a), FCGR3B (CD16b), FUT4 (CD15), IL15RA (CD215), IL1R1 (CD121a), ITGAL (CD11a, Integrin alpha L), ITGAM (CD11b, Integrin alpha M, Mac-1), ITGAX (CD11c, Integrin alpha X), Lamp2 (CD107b, Mac3), LGALS3 (MAC2), PTPRC (CD45), SLAMF7 (CD319), TLR2 (CD282), TLR4 (CD284), TNFRSF1B (CD120b), CD163, CD1A, CD1B, CD209, CD226, CD302 (DCL-1), CD68, CD93, CLEC4A (Dectin-1), FCER2 (CD23), IL1R2 (CDI21b), IL27RA, and MRCI (CD206).

27-28. (canceled)

29. A method of screening for a miRNA agent that modulates inflammation, the method comprising:
a) providing an indicator cell comprising a human genome;
b) contacting the indicator cell with a test miRNA agent; and
c) determining the activity of at least one inflammation regulator in the indicator cell in the presence and absence of the miRNA agent, wherein a change in the activity of the inflammation regulator in the presence of the test miRNA agent identifies the test miRNA agent as a miRNA agent that modulates inflammation.

30-32. (canceled)

33. A composition comprising an agomir or antagomir that modulates the activity of at least one inflammation regulator in a cell and a targeting moiety.

34. The agomir or antagomir of claim 33, wherein the miRNA agent is a miRNA selected from the group consisting of the miRNA is selected from the group consisting of hsa-let-7a-5p, hsa-let-7b-5p, hsa-let-7c, hsa-let-7d-3p, hsa-let-7d-5p, hsa-let-7e-5p, hsa-let-7f-5p, hsa-let-7g-5p, hsa-let-7i-5p, hsa-miR-1, hsa-miR-10a-5p, hsa-miR-100-5p, hsa-miR-103a-2-5p, hsa-miR-103a-3p, hsa-miR-106a-5p, hsa-miR-106b-5p, hsa-miR-107, hsa-miR-122-5p, hsa-miR-124-3p, hsa-miR-1246, hsa-miR-1249, hsa-miR-125a-5p, hsa-miR-125b-5p, hsa-miR-126-3p, hsa-miR-1268a, hsa-miR-1275, hsa-miR-128-3p, hsa-miR-1281, hsa-miR-1307-3p, hsa-miR-130a-3p, hsa-miR-130b-3p, hsa-miR-132-3p, hsa-miR-132-5p, hsa-miR-133a-5p, hsa-miR-133b, hsa-miR-134-5p, hsa-miR-135a-5p, hsa-miR-138-5p, hsa-miR-139-3p, hsa-miR-140-3p, hsa-miR-143-3p, hsa-miR-145-5p, hsa-miR-1469, hsa-miR-146a-5p, hsa-miR-146b-5p, hsa-miR-147a, hsa-miR-148a-3p, hsa-miR-149-5p, hsa-miR-150-3p, hsa-miR-150-5p, hsa-miR-15 la-3p, hsa-miR-151a-5p, hsa-miR-152-3p, hsa-miR-155-5p, hsa-miR-15a-5p, hsa-miR-15b-5p, hsa-miR-16-5p, hsa-miR-17-5p, hsa-miR-181a-3p, hsa-miR-181a-5p, hsa-miR-181b-3p, hsa-miR-181b-5p, hsa-miR-181c-5p, hsa-miR-182-5p, hsa-miR-183-5p, hsa-miR-185-5p, hsa-miR-186-5p, hsa-miR-188-5p, hsa-miR-190b, hsa-miR-191-5p, hsa-miR-1915-3p, hsa-miR-192-5p, hsa-miR-193a-5p, hsa-miR-193b-3p, hsa-miR-197-3p, hsa-miR-19a-3p, hsa-miR-19b-3p, hsa-miR-206, hsa-miR-20a-5p, hsa-miR-21-5p, hsa-miR-210-5p, hsa-miR-212-3p, hsa-miR-214-3p, hsa-miR-22-3p, hsa-miR-221-3p, hsa-miR-222-3p, hsa-miR-223-3p, hsa-miR-23a-3p, hsa-miR-23a-5p, hsa-miR-23b-3p, hsa-miR-24-2-5p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-296-5p, hsa-miR-299-5p, hsa-miR-29a-3p, hsa-miR-29b-3p, hsa-miR-29c-5p, hsa-miR-2c-3p, hsa-miR-30a-5p, hsa-miR-30b-5p, hsa-miR-30c-5p, hsa-miR-30d-5p, hsa-miR-31-5p, hsa-miR-320a, hsa-miR-320b, hsa-miR-320c, hsa-miR-320d, hsa-miR-323a-3p, hsa-miR-324-3p, hsa-miR-324-5p, hsa-miR-326, hsa-miR-328-3p, hsa-miR-330-3p, hsa-miR-331-3p, hsa-miR-337-3p, hsa-miR-337-5p, hsa-miR-339-3p, hsa-miR-339-5p, hsa-miR-33a-5p, hsa-miR-342-3p, hsa-miR-345-5p, hsa-miR-34a-5p, hsa-miR-361-5p, hsa-miR-362-5p, hsa-miR-369-3p, hsa-miR-370-3p, hsa-miR-371a-3p, hsa-miR-374a-5p, hsa-miR-378a-3p, hsa-miR-378a-5p, hsa-miR-421, hsa-miR-423-5p, hsa-miR-424-5p, hsa-miR-425-3p, hsa-miR-425-5p, hsa-miR-432-5p, hsa-miR-433-3p, hsa-miR-483-3p, hsa-miR-483-5p, hsa-miR-484, hsa-miR-485-3p, hsa-miR-487a-3p, hsa-miR-498, hsa-miR-500a-3p, hsa-miR-502-3p, hsa-miR-502-5p, hsa-miR-503-5p, hsa-miR-505-3p, hsa-miR-505-5p, hsa-miR-511-3p, hsa-miR-516a-3p, hsa-miR-517-5p, hsa-miR-517c-3p, hsa-miR-518e-3p, hsa-miR-518f-3p, hsa-miR-519b-3p, hsa-miR-519d-3p, hsa-miR-522-3p, hsa-miR-532-3p, hsa-miR-532-5p, hsa-miR-543, hsa-miR-550a-3p, hsa-miR-572, hsa-miR-573, hsa-miR-574-3p, hsa-miR-576-3p, hsa-miR-582-5p, hsa-miR-604, hsa-miR-610, hsa-miR-618, hsa-miR-622, hsa-miR-625-5p, hsa-miR-629-3p, hsa-miR-629-5p, hsa-miR-630, hsa-miR-638, hsa-miR-642a-5p, hsa-miR-643, hsa-miR-650, hsa-miR-652-3p, hsa-miR-656-3p, hsa-miR-663a, hsa-miR-671-3p, hsa-miR-671-5p, hsa-miR-7-5p, hsa-miR-744-5p, hsa-miR-766-3p, hsa-miR-875-5p, hsa-miR-877-5p, hsa-miR-885-5p, hsa-miR-9-5p, hsa-miR-92a-3p, hsa-miR-92b-3p, hsa-miR-93-5p, hsa-miR-940, hsa-miR-941, hsa-miR-942-5p, hsa-miR-96-5p, hsa-miR-99a-5p, and hsa-miR-99b-5p or an analog, agomir, or antagomir thereof.

35. The agomir or antagomir of claim 33, wherein the miRNA agent is a miRNA selected from the group consisting of the miRNA is selected from the group consisting of hsa-let-7a-5p, hsa-let-7b-5p, hsa-let-7c, hsa-let-7d-3p, hsa-let-7d-5p, hsa-let-7e-5p, hsa-let-7f-5p, hsa-let-7g-5p, hsa-let-7i-5p, hsa-miR-1, hsa-miR-100-5p, hsa-miR-103a-2-5p, hsa-miR-103a-3p, hsa-miR-106a-5p, hsa-miR-106b-5p, hsa-miR-107, hsa-miR-10a-5p, hsa-miR-122-5p, hsa-miR-124-3p, hsa-miR-1246, hsa-miR-1249, hsa-miR-125a-5p, hsa-miR-125b-5p, hsa-miR-126-3p, hsa-miR-1268a, hsa-miR-1275, hsa-miR-130a-3p, hsa-miR-130b-3p, hsa-miR-132-3p, hsa-miR-132-5p, hsa-miR-133b, hsa-miR-135a-5p, hsa-miR-138-5p, hsa-miR-139-3p, hsa-miR-140-3p, hsa-miR-143-3p, hsa-miR-145-5p, hsa-miR-146a-5p, hsa-miR-146b-5p, hsa-miR-147a, hsa-miR-148a-3p, hsa-miR-149-5p, hsa-miR-150-3p, hsa-miR-150-5p, hsa-miR-151a-3p, hsa-miR-151a-5p, hsa-miR-155-5p, hsa-miR-15a-5p, hsa-miR-15b-5p, hsa-miR-16-5p, hsa-miR-17-5p, hsa-miR-181a-3p, hsa-miR-181a-5p, hsa-miR-181b-5p, hsa-miR-181c-5p, hsa-miR-182-5p, hsa-miR-183-5p, hsa-miR-185-5p, hsa-miR-186-5p, hsa-miR-188-5p, hsa-miR-190b, hsa-miR-191-5p, hsa-miR-1915-3p, hsa-miR-192-5p, hsa-miR-193a-5p, hsa-miR-193b-3p, hsa-miR-197-3p, hsa-miR-19a-3p, hsa-miR-19b-3p, hsa-miR-206, hsa-miR-20a-5p, hsa-miR-21-5p, hsa-miR-212-3p, hsa-miR-214-3p, hsa-miR-22-3p, hsa-miR-221-3p, hsa-miR-222-3p, hsa-miR-223-3p, hsa-miR-23a-3p, hsa-miR-23a-5p, hsa-miR-23b-3p, hsa-miR-24-2-5p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-296-5p, hsa-miR-299-5p, hsa-miR-29a-3p, hsa-miR-29b-3p, hsa-miR-29c-5p, hsa-miR-30a-5p, hsa-miR-30b-5p, hsa-miR-30c-5p, hsa-miR-30d-5p, hsa-miR-31-5p, hsa-miR-320a, hsa-miR-320b, hsa-miR-320c, hsa-miR-323a-3p, hsa-miR-324-3p, hsa-miR-324-5p, hsa-miR-326, hsa-miR-330-3p, hsa-miR-331-3p, hsa-miR-337-3p, hsa-miR-337-5p, hsa-miR-339-3p, hsa-miR-339-5p, hsa-miR-33a-5p, hsa-miR-342-3p, hsa-miR-345-5p, hsa-miR-34a-5p, hsa-miR-361-5p, hsa-miR-362-5p, hsa-miR-369-3p, hsa-miR-371a-3p, hsa-miR-374a-5p, hsa-miR-378a-3p, hsa-miR-378a-5p, hsa-miR-421, hsa-miR-423-5p, hsa-miR-424-5p, hsa-miR-425-3p, hsa-miR-425-5p, hsa-miR-432-5p, hsa-miR-483-3p, hsa-miR-483-5p, hsa-miR-484, hsa-miR-498, hsa-miR-500a-3p, hsa-miR-502-3p, hsa-miR-502-5p, hsa-miR-503-5p, hsa-miR-505-3p, hsa-miR-505-5p, hsa-miR-518e-3p, hsa-miR-518f-3p, hsa-miR-532-3p, hsa-miR-532-5p, hsa-miR-543, hsa-miR-550a-3p, hsa-miR-572, hsa-miR-574-3p, hsa-miR-576-3p, hsa-miR-582-5p, hsa-miR-618, hsa-miR-625-5p, hsa-miR-629-3p, hsa-miR-629-5p, hsa-miR-630, hsa-miR-638, hsa-miR-642a-5p, hsa-miR-643, hsa-miR-650, hsa-miR-652-3p, hsa-miR-663a, hsa-miR-671-5p, hsa-miR-7-5p, hsa-miR-744-5p, hsa-miR-877-5p, hsa-miR-9-5p, hsa-miR-92a-3p, hsa-miR-92b-3p, hsa-miR-93-5p, hsa-miR-940, hsa-miR-941, hsa-miR-96-5p, hsa-miR-99a-5p, and hsa-miR-99b-5p or an analog, agomir, or antagomir thereof.

36. The agomir or antagomir of claim 33, wherein the miRNA agent is a miRNA selected from the group consisting of the miRNA is selected from the group consisting of hsa-let-7a-5p, hsa-let-7b-5p, hsa-let-7c, hsa-let-7e-5p, hsa-let-7f-5p, hsa-let-7g-5p, hsa-let-7i-5p, hsa-miR-106a-5p, hsa-miR-107, hsa-miR-125a-5p, hsa-miR-1275, hsa-miR-130a-3p, hsa-miR-130b-3p, hsa-miR-15a-5p, hsa-miR-15b-5p, hsa-miR-16-5p, hsa-miR-17-5p, hsa-miR-185-5p, hsa-miR-188-5p, hsa-miR-193a-5p, hsa-miR-206, hsa-miR-20a-5p, hsa-miR-296-5p, hsa-miR-30d-5p, hsa-miR-320a, hsa-miR-320b, hsa-miR-320c, hsa-miR-324-3p, hsa-miR-326, hsa-miR-330-3p, hsa-miR-331-3p, hsa-miR-339-5p, hsa-miR-369-3p, hsa-miR-423-5p, hsa-miR-424-5p, hsa-miR-484, hsa-miR-498, hsa-miR-532-3p, hsa-miR-532-5p, hsa-miR-543, hsa-miR-650, hsa-miR-671-5p, hsa-miR-93-5p, and hsa-miR-940 or an analog, agomir, or antagomir thereof.

37. The agomir or antagomir of claim 33, wherein the miRNA agent is a miRNA selected from the group consisting of the miRNA is selected from the group consisting of hsa-let-7a-5p, hsa-let-7b-5p, hsa-let-7c, hsa-let-7e-5p, hsa-let-7f-5p, hsa-let-7i-5p, hsa-miR-1, hsa-miR-106a-5p, hsa-miR-107, hsa-miR-125a-5p, hsa-miR-1275, hsa-miR-130a-3p, hsa-miR-130b-3p, hsa-miR-140-3p, hsa-miR-146b-5p, hsa-miR-15a-5p, hsa-miR-15b-5p, hsa-miR-16-5p, hsa-miR-17-5p, hsa-miR-186-5p, hsa-miR-188-5p, hsa-miR-206, hsa-miR-20a-5p, hsa-miR-27a-3p, hsa-miR-27b-3p, hsa-miR-28-3p, hsa-miR-30b-5p, hsa-miR-30d-5p, hsa-miR-320a, hsa-miR-320b, hsa-miR-320c, hsa-miR-326, hsa-miR-330-3p, hsa-miR-342-3p, hsa-miR-361-5p, hsa-miR-362-5p, hsa-miR-423-5p, hsa-miR-424-5p, hsa-miR-498, hsa-miR-532-5p, hsa-miR-543, hsa-miR-618, hsa-miR-650, hsa-miR-671-5p, hsa-miR-93-5p, hsa-miR-940 or an analog, agomir, or antagomir thereof.

38. The agomir or antagomir of claim 33, wherein the miRNA agent is a miRNA selected from the group consisting of the miRNA is selected from the group consisting of hsa-let-7a-5p, hsa-let-7b-5p, hsa-let-7c, hsa-let-7e-5p, hsa-let-7f-5p, hsa-let-7i-5p, hsa-miR-106a-5p, hsa-miR-107, hsa-miR-125a-5p, hsa-miR-1275, hsa-miR-130a-3p, hsa-miR-130b-3p, hsa-miR-15a-5p, hsa-miR-15b-5p, hsa-miR-16-5p, hsa-miR-17-5p, hsa-miR-188-5p, hsa-miR-206, hsa-miR-20a-5p, hsa-miR-30d-5p, hsa-miR-320a, hsa-miR-320b, hsa-miR-320c, hsa-miR-326, hsa-miR-330-3p, hsa-miR-423-5p, hsa-miR-424-5p, hsa-miR-498, hsa-miR-532-5p, hsa-miR-543, hsa-miR-650, hsa-miR-671-5p, hsa-miR-93-5p, and hsa-miR-940 or an analog, agomir, or antagomir thereof.

39-45. (canceled)
